(19) United States
(12) Patent Application Publication
(10) Pub. No.: US 2026/0153441 A1
(43) Pub. Date: Jun. 4, 2026

(54) METHODS AND SYSTEMS FOR MICROBIAL DETECTION USING RAMAN SPECTROSCOPY

(71) Applicant: JANSSEN PHARMACEUTICA NV, Turnhoutseweg 30 (BE)

(72) Inventors: James HAUSCHILD, Raritan, NJ (US); Karin BALSS, Spring House, PA (US); Olav LYNGBERG, Titusville, NJ (US); Emily CURTIS, Spring House, PA (US); Erin LESTER, Spring House, PA (US); Hassaan SHEIKH, Somerset, NJ (US)

(21) Appl. No.: 19/122,598

(22) PCT Filed: Oct. 20, 2023
(86) PCT No.: PCT/IB2023/060638

§ 371 Date: Apr. 18, 2025

Related U.S. Application Data
(60) Provisional application No. 63/380,446, filed on Oct. 21, 2022.
(60) Provisional application No. 63/380,455, filed on Oct. 21, 2022.
(60) Provisional application No. 63/380,461, filed on Oct. 21, 2022.
(60) Provisional application No. 63/380,494, filed on Oct. 21, 2022.
(60) Provisional application No. 63/380,505, filed on Oct. 21, 2022.
(60) Provisional application No. 63/380,511, filed on Oct. 21, 2022.

(51) Int. Cl. G01N 21/64; G01N 21/65; G01N 21/76; G01N 33/569

(52) U.S. Cl. CPC G01N 21/6428; G01N 21/65; G01N 21/763; G01N 33/569; G01N 2021/6439

(57) ABSTRACT

Provided is a method for detecting a microbe in a sample comprising measuring a Raman spectrum of the sample. In some embodiments, the measuring is automated. In some embodiments, the monitoring is continuous and/or non-invasive and/or non-destructive. Also provided is a system for detecting a microbe in a sample comprising a means for measuring a Raman spectrum of the sample. In some embodiments, the measuring is automated. In some embodiments, the monitoring is continuous and/or non-invasive and/or non-destructive. Also provided are methods and systems for assaying a test agent.

### CROSS REFERENCE TO RELATED APPLICATIONS

This application is entitled to priority under 35 U.S.C. § 119 (e) to U.S. Provisional Application Nos. 63/380,446, filed Oct. 21, 2022, 63/380,455, filed Oct. 21, 2022, 63/380,461, filed Oct. 21, 2022, 63/380,494, filed Oct. 21, 2022, 63/380,505, filed Oct. 21, 2022, 63/380,511, filed Oct. 21, 2022, each of which is hereby incorporated by reference in its entirety.

### FIELD

The general inventive concepts relate to the field of microbial detection and more particularly to methods and systems for the automated detection of microbes using Raman spectroscopy.

### BACKGROUND

During the manufacture of biopharmaceuticals the entire process and resulting drug substance and drug product must maintain set bioburden limits. Bioburden testing is routinely performed as an in-process control (IPC) test for drug substances and for final product release of nonsterile drug product. Multiple unit operations in Biologics manufacturing such as the production bioreactor, chromatography purification, and filtration steps are monitored for bioburden as in-process control (IPC) tests to assure microbiological quality and sterility assurance. Conventional microbiological testing is based on 100 year-old technology using agar plates for enumerating microbial colony-forming units (CFU).¹⁻² The conventional plate count for microbial bioburden monitoring of in-process or finished product samples requires 2-5 days of incubation for quantitative enumeration. Conventional microbiology methods are manual, subject to cross contamination, provide slow retrospective results, are open to subjective interpretation, have limited data traceability and have the potential for data integrity issues. Current bioreactor specifications allow for low level of CFUs (<5 CFU/0.5 mL) to avoid false positives due to errors in compendial testing. Frequently, to avoid unnecessary delays, the manufacturing process proceeds at risk to the next unit operation pending IPC bioburden testing. Finally, the DS/DP release testing requirements places enormous demand on the quality control laboratory to conduct thousands of tests per year and represents one bottleneck to final release of product.

Biopharmaceutical manufacturing companies are challenged to increase efficiency in manufacturing to meet the demands of patients for life saving therapies. Batch release testing for biopharmaceuticals can take up to 30 days, primarily due to the lengthy time it takes to complete microbiological quality and sterility assurance testing, such as mycoplasma, bioburden, sterility and endotoxin.³ Efforts in small molecule API pharmaceuticals have successfully incorporated multivariate statistical modeling and process analytical technology (PAT) to enable measurements of both IPCs (i.e. content uniformity) and final DP release testing (i.e. dissolution and assay) enabling release of product within hours of manufacture, effectively achieving real time release.⁴⁻⁵ Real-time release testing (RTRT) is defined as “the ability to evaluate and ensure the quality of in-process and/or final drug product based on process data, which typically includes a valid combination of measured material attributes and process controls”. In biopharmaceuticals, efforts are underway to apply the same approach to eliminate IPCs and reduce quality control (QC) drug substance and drug product release testing. Other than bioburden, common examples of IPCs in bioreactor operations include glucose, viable cell density, and titer, all of which have been successfully measured with in-line PAT sensors.⁶⁻¹⁴ Examples of spectroscopy-based PAT sensing for in-line testing of quality attributes required for drug substance and drug product release include protein and excipient concentration, and protein-specific critical quality attributes such as charge, aggregation, glycation, or post-translational modifications PTMs.¹⁵⁻¹⁸ Recently, there is increased interest in developing new methods to address bioburden and sterility testing for IPC and release.¹⁹⁻²² To date, these efforts are focused on development of assays that decrease testing time significantly, identify species, and are either intended for the QC laboratory or at-line testing. Most of these tests are destructive and all of them require sample preparation (labeling, filtering, or other isolation) prior to testing. A recent effort requires centrifugation of a sample prior to Raman spectroscopy analysis.²⁵

There remains a need for methods and systems for the rapid, automated detection of microbes using Raman spectroscopy.

### SUMMARY

### Methods

### Methods for Detecting a Microbe

Provided is a method for detecting a microbe in a sample comprising: measuring a Raman spectrum of the sample.

In some embodiments, the sample is exposed to a coherent light source prior to measuring the Raman spectrum of the sample.

In some embodiments, the Raman spectrum is measured using a Raman spectrometer.

In some embodiments, the Raman spectrometer comprises a laser. In further embodiments, the laser is a multimode diode laser.

In some embodiments, the laser wavelength is from 300 nm to 1200 nm, from 350 nm to 1100 nm, from 400 nm to 1100 nm, from 400 nm to 1064 nm, from 450 nm to 1064 nm, from 500 nm to 1064 nm, from 550 nm to 1064 nm, from 600 nm to 1064 nm, from 650 nm to 1064 nm, from 700 nm to 1064 nm, from 450 nm to 1100 nm, or from 500 nm to 1100 nm. In any embodiment, the light source is a narrow bandwidth laser with a wavelength of about 800 nm, about 785 nm, about 750 nm, about 725 nm, about 700 nm, about 675 nm, about 650 nm, about 625 nm, about 600 nm, about 575 nm, about 550 nm, about 532 nm, about 525 nm, or about 500 nm.

In some embodiments, the laser wavelength is 785 nm.

In some embodiments, the sample is in a bioreactor, a harvest tank, a chromatography skid, a chromatography column, an ultrafiltration (UF) skid, a diafiltration (DF) skid, a UF/DF skid, a fill finish tank or an incubator.

In some embodiments, the Raman spectrum of the sample is compared to the Raman spectrum of a control sample.

In some embodiments, the spectral range of the sample is from about 100 to about 3600 cm⁻¹. In further embodiments, the spectral range of the sample is from about 200 to about 3600 cm⁻¹. In further embodiments, the spectral range of the sample is from about 425 to about 1800 cm⁻¹. In yet further embodiments, the spectral range of the sample is from about 2800 to about 3100 cm⁻¹.

In some embodiments, the spectral range of the control sample is from about 100 to about 3600 cm⁻¹. In further embodiments, the spectral range of the control sample is from about 200 to about 3600 cm⁻¹. In further embodiments, the spectral range of the control sample is from about 425 to about 1800 cm⁻¹. In yet further embodiments, the spectral range of the control sample is from about 2800 to about 3100 cm⁻¹.

In some embodiments, the Raman spectrum of the sample is monitored over a period of time.

In some embodiments, the monitoring is automated.

In some embodiments, the monitoring is continuous.

In some embodiments, the monitoring is non-invasive and/or non-destructive.

In some embodiments, the sample has been contacted with an antibody to the microbe.

In some embodiments, the sample is contacted with a fluorescent agent or a bioluminescent agent.

In some embodiments, the method further comprises detecting fluorescence of the sample. In further embodiments, the means for detecting fluorescence is a fluorimeter.

In some embodiments, the method further comprises detecting bioluminescence of the sample. In further embodiments, the means for detecting bioluminescence is a luminometer.

In some embodiments, the microbe includes but is not limited to Cutibacterium acnes, Staphylococcus aureus, Aspergillus brasiliensis, Candida albicans, Bacillus subtilis, Escherichia coli, Pseudomonas aeruginosa, Staphylococcus epidermidis, Streptococcus pyogenes, Micrococcus luteus, Burkholderia cepacia, Salmonella typhimurium, or Bacillus cereus. In some embodiments, the microbe is Cutibacterium acnes, Staphylococcus aureus, Aspergillus brasiliensis, Candida albicans, Bacillus subtilis, Escherichia coli, Pseudomonas aeruginosa, Staphylococcus epidermidis, Streptococcus pyogenes, Micrococcus luteus, Burkholderia cepacia, Salmonella typhimurium, or Bacillus cereus.

In some embodiments, the measuring of the Raman spectrum is automated.

In some embodiments, the Raman spectrum is subjected to data analysis.

In some embodiments, data analysis of the Raman spectrum comprises modeling performed using principal component analysis-X (PCA-X), orthogonal partial least squares (OPLS), k-nearest neighbors (KNN), orthogonal partial least squares discriminant analysis (OPLS-DA), partial least squares discriminant analysis (PLS-DA), or combinations thereof. In some embodiments, the modeling is discriminant modeling.

In some embodiments, the method further comprises incubation with D2O for assessment of microbial viability. In further embodiments, a sample from a cell culture vessel is diverted to a flow cell chamber for Raman analysis. In some embodiments, the derivatization is performed prior to measuring the Raman spectrum of the sample.

In some embodiments, the sample comprises a eukaryotic cell.

In some embodiments, the eukaryotic cell produces a protein, an antibody or fragment thereof, a duobody, a receptor, a chimeric antigen receptor, a glycoprotein, a viral vector, or a combination thereof.

In some embodiments, the eukaryotic cell is a mouse cell, a chinese hamster ovary (CHO) cell, or a human cell. In some embodiments, the cell is a T cell, or a B cell. In some embodiments, the mouse cell is a mouse Sp2/0 cell. In some embodiments, the cell is HEK293F. In some embodiments, the cell is PER.C6.

In some embodiments, the eukaryotic cell is a chimeric antigen receptor T cell (CAR-T cell).

In some embodiments, the sample is not centrifuged prior to measuring the Raman spectrum of the sample.

In some embodiments, the microbe is actively growing.

### Methods for Continuous Monitoring

Provided is a method for continuous monitoring for microbial presence in a sample comprising: measuring a Raman spectrum of the sample.

In some embodiments, the Raman spectrum of the sample is monitored over a period of time.

In some embodiments, the monitoring is non-invasive and/or non-destructive.

In some embodiments, the sample is exposed to a coherent light source prior to measuring the Raman spectrum of the sample.

In some embodiments, the Raman spectrum is measured using a Raman spectrometer.

In some embodiments, the Raman spectrometer comprises a laser. In further embodiments, the laser is a multimode diode laser.

In some embodiments, the laser wavelength is from 300 nm to 1200 nm, from 350 nm to 1100 nm, from 400 nm to 1100 nm, from 400 nm to 1064 nm, from 450 nm to 1064 nm, from 500 nm to 1064 nm, from 550 nm to 1064 nm, from 600 nm to 1064 nm, from 650 nm to 1064 nm, from 700 nm to 1064 nm, from 450 nm to 1100 nm, or from 500 nm to 1100 nm. In any embodiment, the light source is a narrow bandwidth laser with a wavelength of about 800 nm, about 785 nm, about 750 nm, about 725 nm, about 700 nm, about 675 nm, about 650 nm, about 625 nm, about 600 nm, about 575 nm, about 550 nm, about 532 nm, about 525 nm, or about 500 nm.

In some embodiments, the laser wavelength is 785 nm.

In some embodiments, the sample is in a bioreactor, a harvest tank, a chromatography skid, a chromatography column, an ultrafiltration (UF) skid, a diafiltration (DF) skid, a UF/DF skid, a fill finish tank or an incubator.

In some embodiments, the Raman spectrum of the sample is compared to the Raman spectrum of a control sample.

In some embodiments, the spectral range of the sample is from about 100 to about 3600 cm⁻¹. In further embodiments, the spectral range of the sample is from about 200 to about 3600 cm⁻¹. In further embodiments, the spectral range of the sample is from about 425 to about 1800 cm⁻¹. In yet further embodiments, the spectral range of the sample is from about 2800 to about 3100 cm⁻¹.

In some embodiments, the spectral range of the control sample is from about 100 to about 3600 cm⁻¹. In further embodiments, the spectral range of the control sample is from about 200 to about 3600 cm⁻¹. In further embodiments, the spectral range of the control sample is from about 425 to about 1800 cm⁻¹. In yet further embodiments, the spectral range of the control sample is from about 2800 to about 3100 cm⁻¹.

In some embodiments, the monitoring is automated.

In some embodiments, the monitoring is non-invasive and/or non-destructive.

In further embodiments, the sample has been contacted with an antibody to the microbe.

In some embodiments, the sample has been contacted with a fluorescent agent or a bioluminescent agent. In some embodiments, the method further comprises detecting fluorescence or bioluminescence of the sample.

In some embodiments, the microbe includes but is not limited to Cutibacterium acnes, Staphylococcus aureus, Aspergillus brasiliensis, Candida albicans, Bacillus subtilis, Escherichia coli, Pseudomonas aeruginosa, Staphylococcus epidermidis, Streptococcus pyogenes, Micrococcus luteus, Burkholderia cepacia, Salmonella typhimurium, or Bacillus cereus. In some embodiments, the microbe is Cutibacterium acnes, Staphylococcus aureus, Aspergillus brasiliensis, Candida albicans, Bacillus subtilis, Escherichia coli, Pseudomonas aeruginosa, Staphylococcus epidermidis, Streptococcus pyogenes, Micrococcus luteus, Burkholderia cepacia, Salmonella typhimurium, or Bacillus cereus.

In some embodiments, the measuring of the Raman spectrum is automated.

In some embodiments, the Raman spectrum is subjected to data analysis.

In some embodiments, data analysis of the Raman spectrum comprises discriminant modeling performed using principal component analysis-X (PCA-X), orthogonal partial least squares (OPLS), k-nearest neighbors (KNN), orthogonal partial least squares discriminant analysis (OPLS-DA), partial least squares discriminant analysis (PLS-DA), or combinations thereof.

In some embodiments, the method further comprises incubation with D2O for assessment of microbial viability. In further embodiments, a sample from a cell culture vessel is diverted to a flow cell chamber for Raman analysis. In some embodiments, the derivatization is performed prior to measuring the Raman spectrum of the sample.

In some embodiments, the sample comprises a eukaryotic cell.

In some embodiments, the eukaryotic cell produces a protein, an antibody, a duobody, a receptor, a chimeric antigen receptor, a glycoprotein, a viral vector, or a combination thereof.

In some embodiments, the eukaryotic cell is a mouse cell, a chinese hamster ovary (CHO) cell, or a human cell. In some embodiments, the cell is a T cell, or a B cell. In some embodiments, the mouse cell is a mouse Sp2/0 cell. In some embodiments, the cell is HEK293F. In some embodiments, the cell is PER.C6.

In some embodiments, the eukaryotic cell is a chimeric antigen receptor T cell (CAR-T cell).

In some embodiments, the sample is not centrifuged prior to measuring the Raman spectrum of the sample.

In some embodiments, the microbe is actively growing.

### Methods for Antimicrobial Testing

Provided is a method for assaying a test agent comprising: adding a test agent to a sample comprising a microbe; and measuring a Raman spectrum of the sample.

In some embodiments, the sample is exposed to a coherent light source prior to measuring the Raman spectrum of the sample.

In some embodiments, the Raman spectrum is measured using a Raman spectrometer.

In some embodiments, the Raman spectrometer comprises a laser. In further embodiments, the laser is a multimode diode laser.

Suitable laser wavelengths include, without limitation, wavelengths from 300 nm to 1200 nm, from 350 nm to 1100 nm, from 400 nm to 1100 nm, from 400 nm to 1064 nm, from 450 nm to 1064 nm, from 500 nm to 1064 nm, from 550 nm to 1064 nm, from 600 nm to 1064 nm, from 650 nm to 1064 nm, from 700 nm to 1064 nm, from 450 nm to 1100 nm, or from 500 nm to 1100 nm. In any embodiment, the light source is a narrow bandwidth laser with a wavelength of about 800 nm, about 785 nm, about 750 nm, about 725 nm, about 700 nm, about 675 nm, about 650 nm, about 625 nm, about 600 nm, about 575 nm, about 550 nm, about 532 nm, about 525 nm, or about 500 nm.

In some embodiments, the laser wavelength is 785 nm.

In some embodiments, the sample is in a bioreactor, a harvest tank, a chromatography skid, a chromatography column, an ultrafiltration (UF) skid, a diafiltration (DF) skid, a UF/DF skid, a fill finish tank or an incubator.

In some embodiments, the Raman spectrum of the sample is compared to the Raman spectrum of a control sample.

In some embodiments, the spectral range of the sample is from about 100 to about 3600 cm⁻¹. In further embodiments, the spectral range of the sample is from about 200 to about 3600 cm⁻¹. In further embodiments, the spectral range of the sample is from about 425 to about 1800 cm⁻¹. In yet further embodiments, the spectral range of the sample is from about 2800 to about 3100 cm⁻¹.

In some embodiments, the spectral range of the control sample is from about 100 to about 3600 cm⁻¹. In further embodiments, the spectral range of the control sample is from about 200 to about 3600 cm⁻¹. In further embodiments, the spectral range of the control sample is from about 425 to about 1800 cm⁻¹. In yet further embodiments, the spectral range of the control sample is from about 2800 to about 3100 cm⁻¹.

In some embodiments, the Raman spectrum of the sample is monitored over a period of time.

In some embodiments, the monitoring is automated.

In some embodiments, the monitoring is continuous.

In some embodiments, the monitoring is non-invasive and/or non-destructive.

In some embodiments, the test agent is a compound, a supplement, an antibiotic, a bacteriophage, or a combination thereof.

In some embodiments, the sample has been contacted with an antibody to the microbe.

In some embodiments, the sample is contacted with a fluorescent agent or a bioluminescent agent.

In some embodiments, the method further comprises detecting fluorescence of the sample. In further embodiments, the means for detecting fluorescence is a fluorimeter.

In some embodiments, the method further comprises detecting bioluminescence of the sample. In further embodiments, the means for detecting bioluminescence is a luminometer.

In some embodiments, the microbe includes but is not limited to Cutibacterium acnes, Staphylococcus aureus, Aspergillus brasiliensis, Candida albicans, Bacillus subtilis, Escherichia coli, Pseudomonas aeruginosa, Staphylococcus epidermidis, Streptococcus pyogenes, Micrococcus luteus, Burkholderia cepacia, Salmonella typhimurium, or Bacillus cereus. In some embodiments, the microbe is Cutibacterium acnes, Staphylococcus aureus, Aspergillus brasiliensis, Candida albicans, Bacillus subtilis, Escherichia coli, Pseudomonas aeruginosa, Staphylococcus epidermidis, Streptococcus pyogenes, Micrococcus luteus, Burkholderia cepacia, Salmonella typhimurium, or Bacillus cereus.

In some embodiments, the measuring of the Raman spectrum is automated.

In some embodiments, the Raman spectrum is subjected to data analysis.

In some embodiments, data analysis of the Raman spectrum comprises modeling performed using principal component analysis-X (PCA-X), orthogonal partial least squares (OPLS), k-nearest neighbors (KNN), orthogonal partial least squares discriminant analysis (OPLS-DA), partial least squares discriminant analysis (PLS-DA), or combinations thereof. In some embodiments, the modeling is discriminant modeling.

In some embodiments, the method further comprises incubation with D2O for assessment of microbial viability. In further embodiments, a sample from a cell culture vessel is diverted to a flow cell chamber for Raman analysis. In some embodiments, the derivatization is performed prior to measuring the Raman spectrum of the sample.

In some embodiments, the sample comprises a eukaryotic cell.

In some embodiments, the eukaryotic cell produces a protein, an antibody, a duobody, a receptor, a chimeric antigen receptor, a glycoprotein, a viral vector, or a combination thereof.

In some embodiments, the eukaryotic cell is a mouse cell, a chinese hamster ovary (CHO) cell, or a human cell. In some embodiments, the cell is a T cell, or a B cell. In some embodiments, the mouse cell is a mouse Sp2/0 cell. In some embodiments, the cell is HEK293F. In some embodiments, the cell is PER.C6.

In some embodiments, the eukaryotic cell is a chimeric antigen receptor T cell (CAR-T cell).

In some embodiments, the sample is not centrifuged prior to measuring the Raman spectrum of the sample.

In some embodiments, the microbe is actively growing.

### Systems

### Systems for Detecting a Microbe

Provided is a system for detecting a microbe in a sample comprising: a means for measuring a Raman spectrum of the sample.

In some embodiments, the system further comprises a coherent light source.

In some embodiments, the means for measuring a Raman spectrum is a Raman spectrometer.

In some embodiments, the Raman spectrometer comprises a laser. In further embodiments, the laser is a multimode diode laser.

In some embodiments, the laser wavelength is from 300 nm to 1200 nm, from 350 nm to 1100 nm, from 400 nm to 1100 nm, from 400 nm to 1064 nm, from 450 nm to 1064 nm, from 500 nm to 1064 nm, from 550 nm to 1064 nm, from 600 nm to 1064 nm, from 650 nm to 1064 nm, from 700 nm to 1064 nm, from 450 nm to 1100 nm, or from 500 nm to 1100 nm. In any embodiment, the light source is a narrow bandwidth laser with a wavelength of about 800 nm, about 785 nm, about 750 nm, about 725 nm, about 700 nm, about 675 nm, about 650 nm, about 625 nm, about 600 nm, about 575 nm, about 550 nm, about 532 nm, about 525 nm, or about 500 nm.

In some embodiments, the laser wavelength is 785 nm.

In some embodiments, the sample is in a bioreactor, a harvest tank, a chromatography skid, a chromatography column, an ultrafiltration (UF) skid, a diafiltration (DF) skid, a UF/DF skid, a fill finish tank or an incubator.

In some embodiments, the Raman spectrum of the sample is compared to the Raman spectrum of a control sample.

In some embodiments, the spectral range of the sample is from about 100 to about 3600 cm⁻¹. In further embodiments, the spectral range of the sample is from about 200 to about 3600 cm⁻¹. In further embodiments, the spectral range of the sample is from about 425 to about 1800 cm⁻¹. In yet further embodiments, the spectral range of the sample is from about 2800 to about 3100 cm⁻¹.

In some embodiments, the spectral range of the control sample is from about 100 to about 3600 cm⁻¹. In further embodiments, the spectral range of the control sample is from about 200 to about 3600 cm⁻¹. In further embodiments, the spectral range of the control sample is from about 425 to about 1800 cm⁻¹. In yet further embodiments, the spectral range of the control sample is from about 2800 to about 3100 cm⁻¹.

In some embodiments, the Raman spectrum of the sample is monitored over a period of time.

In some embodiments, the monitoring is automated.

In some embodiments, the monitoring is continuous.

In some embodiments, the monitoring is non-invasive and/or non-destructive.

In some embodiments, the sample has been contacted with an antibody to the microbe.

In some embodiments, the sample is contacted with a fluorescent agent or a bioluminescent agent.

In some embodiments, the system further comprises a means for detecting fluorescence of the sample. In further embodiments, the means for detecting fluorescence is a fluorimeter.

In some embodiments, the system further comprises a means for detecting bioluminescence of the sample. In further embodiments, the means for detecting bioluminescence is a luminometer.

In some embodiments, the microbe includes but is not limited to Cutibacterium acnes, Staphylococcus aureus, Aspergillus brasiliensis, Candida albicans, Bacillus subtilis, Escherichia coli, Pseudomonas aeruginosa, Staphylococcus epidermidis, Streptococcus pyogenes, Micrococcus luteus, Burkholderia cepacia, Salmonella typhimurium, or Bacillus cereus. In some embodiments, the microbe is Cutibacterium acnes, Staphylococcus aureus, Aspergillus brasiliensis, Candida albicans, Bacillus subtilis, Escherichia coli, Pseudomonas aeruginosa, Staphylococcus epidermidis, Streptococcus pyogenes, Micrococcus luteus, Burkholderia cepacia, Salmonella typhimurium, or Bacillus cereus.

In some embodiments, the measuring of the Raman spectrum is automated.

In some embodiments, the Raman spectrum is subjected to data analysis.

In some embodiments, data analysis of the Raman spectrum comprises modeling performed using principal component analysis-X (PCA-X), orthogonal partial least squares (OPLS), k-nearest neighbors (KNN), orthogonal partial least squares discriminant analysis (OPLS-DA), partial least squares discriminant analysis (PLS-DA), or combinations thereof. In some embodiments, the modeling is discriminant modeling.

In some embodiments, the method further comprises incubation with D2O for assessment of microbial viability. In further embodiments, a sample from a cell culture vessel is diverted to a flow cell chamber for Raman analysis. In some embodiments, the derivatization is performed prior to measuring the Raman spectrum of the sample.

In some embodiments, the sample comprises a eukaryotic cell.

In some embodiments, the eukaryotic cell produces a protein, an antibody or fragment thereof, a duobody, a receptor, a chimeric antigen receptor, a glycoprotein, a viral vector, or a combination thereof.

In some embodiments, the eukaryotic cell is a mouse cell, a chinese hamster ovary (CHO) cell, or a human cell. In some embodiments, the cell is a T cell, or a B cell. In some embodiments, the mouse cell is a mouse Sp2/0 cell. In some embodiments, the cell is HEK293F. In some embodiments, the cell is PER.C6.

In some embodiments, the eukaryotic cell is a chimeric antigen receptor T cell (CAR-T cell).

In some embodiments, the sample is not centrifuged prior to measuring the Raman spectrum of the sample.

In some embodiments, the microbe is actively growing.

### Systems for Continuous Monitoring

Also provided is a system for continuous monitoring for microbial presence in a sample comprising: a means for measuring a Raman spectrum of the sample.

In some embodiments, the Raman spectrum of the sample is monitored over a period of time.

In some embodiments, the monitoring is non-invasive and/or non-destructive.

In some embodiments, the sample is exposed to a coherent light source prior to measuring the Raman spectrum of the sample.

In some embodiments, the Raman spectrum is measured using a Raman spectrometer.

In some embodiments, the Raman spectrometer comprises a laser. In further embodiments, the laser is a multimode diode laser.

In some embodiments, the laser wavelength is from 300 nm to 1200 nm, from 350 nm to 1100 nm, from 400 nm to 1100 nm, from 400 nm to 1064 nm, from 450 nm to 1064 nm, from 500 nm to 1064 nm, from 550 nm to 1064 nm, from 600 nm to 1064 nm, from 650 nm to 1064 nm, from 700 nm to 1064 nm, from 450 nm to 1100 nm, or from 500 nm to 1100 nm. In any embodiment, the light source is a narrow bandwidth laser with a wavelength of about 800 nm, about 785 nm, about 750 nm, about 725 nm, about 700 nm, about 675 nm, about 650 nm, about 625 nm, about 600 nm, about 575 nm, about 550 nm, about 532 nm, about 525 nm, or about 500 nm.

In some embodiments, the laser wavelength is 785 nm.

In some embodiments, the sample is in a bioreactor, a harvest tank, a chromatography skid, a chromatography column, an ultrafiltration (UF) skid, a diafiltration (DF) skid, a UF/DF skid, a fill finish tank or an incubator.

In some embodiments, the Raman spectrum of the sample is compared to the Raman spectrum of a control sample.

In some embodiments, the spectral range of the sample is from about 100 to about 3600 cm⁻¹. In further embodiments, the spectral range of the sample is from about 200 to about 3600 cm⁻¹. In further embodiments, the spectral range of the sample is from about 425 to about 1800 cm⁻¹. In yet further embodiments, the spectral range of the sample is from about 2800 to about 3100 cm⁻¹.

In some embodiments, the spectral range of the control sample is from about 100 to about 3600 cm⁻¹. In further embodiments, the spectral range of the control sample is from about 200 to about 3600 cm⁻¹. In further embodiments, the spectral range of the control sample is from about 425 to about 1800 cm⁻¹. In yet further embodiments, the spectral range of the control sample is from about 2800 to about 3100 cm⁻¹.

In some embodiments, the monitoring is automated.

In some embodiments, the monitoring is non-invasive and/or non-destructive.

In some embodiments, the system further comprises detecting fluorescence of the sample.

In further embodiments, the sample has been contacted with an antibody to the microbe.

In some embodiments, the sample has been contacted with a fluorescent agent or a bioluminescent agent.

In some embodiments, the system further comprises a means for detecting fluorescence of the sample. In further embodiments, the means for detecting fluorescence is a fluorimeter.

In some embodiments, the system further comprises a means for detecting bioluminescence of the sample. In further embodiments, the means for detecting bioluminescence is a luminometer.

In some embodiments, the microbe includes but is not limited to Cutibacterium acnes, Staphylococcus aureus, Aspergillus brasiliensis, Candida albicans, Bacillus subtilis, Escherichia coli, Pseudomonas aeruginosa, Staphylococcus epidermidis, Streptococcus pyogenes, Micrococcus luteus, Burkholderia cepacia, Salmonella typhimurium, or Bacillus cereus. In some embodiments, the microbe is Cutibacterium acnes, Staphylococcus aureus, Aspergillus brasiliensis, Candida albicans, Bacillus subtilis, Escherichia coli, Pseudomonas aeruginosa, Staphylococcus epidermidis, Streptococcus pyogenes, Micrococcus luteus, Burkholderia cepacia, Salmonella typhimurium, or Bacillus cereus.

In some embodiments, the measuring of the Raman spectrum is automated.

In some embodiments, the Raman spectrum is subjected to data analysis.

In some embodiments, data analysis of the Raman spectrum comprises discriminant modeling performed using principal component analysis-X (PCA-X), orthogonal partial least squares (OPLS), k-nearest neighbors (KNN), orthogonal partial least squares discriminant analysis (OPLS-DA), partial least squares discriminant analysis (PLS-DA), or combinations thereof.

In some embodiments, the method further comprises incubation with D2O for assessment of microbial viability. In further embodiments, a sample from a cell culture vessel is diverted to a flow cell chamber for Raman analysis. In some embodiments, the derivatization is performed prior to measuring the Raman spectrum of the sample.

In some embodiments, the sample comprises a eukaryotic cell.

In some embodiments, the eukaryotic cell produces a protein, an antibody, a duobody, a receptor, a chimeric antigen receptor, a glycoprotein, a viral vector, or a combination thereof.

In some embodiments, the eukaryotic cell is a mouse cell, a chinese hamster ovary (CHO) cell, or a human cell. In some embodiments, the cell is a T cell, or a B cell. In some embodiments, the mouse cell is a mouse Sp2/0 cell. In some embodiments, the cell is HEK293F. In some embodiments, the cell is PER.C6.

In some embodiments, the eukaryotic cell is a chimeric antigen receptor T cell (CAR-T cell).

In some embodiments, the sample is not centrifuged prior to measuring the Raman spectrum of the sample.

In some embodiments, the microbe is actively growing.

### Systems for Assaying a Test Agent

Provided is a system for assaying a test agent comprising: a means for measuring a Raman spectrum of a sample to which a test agent has been added.

In some embodiments, the sample is exposed to a coherent light source prior to measuring the Raman spectrum of the sample.

In some embodiments, the Raman spectrum is measured using a Raman spectrometer.

In some embodiments, the Raman spectrometer comprises a laser. In further embodiments, the laser is a multimode diode laser.

Suitable laser wavelengths include, without limitation, wavelengths from 300 nm to 1200 nm, from 350 nm to 1100 nm, from 400 nm to 1100 nm, from 400 nm to 1064 nm, from 450 nm to 1064 nm, from 500 nm to 1064 nm, from 550 nm to 1064 nm, from 600 nm to 1064 nm, from 650 nm to 1064 nm, from 700 nm to 1064 nm, from 450 nm to 1100 nm, or from 500 nm to 1100 nm. In any embodiment, the light source is a narrow bandwidth laser with a wavelength of about 800 nm, about 785 nm, about 750 nm, about 725 nm, about 700 nm, about 675 nm, about 650 nm, about 625 nm, about 600 nm, about 575 nm, about 550 nm, about 532 nm, about 525 nm, or about 500 nm.

In some embodiments, the laser wavelength is 785 nm.

In some embodiments, the sample is in a bioreactor, a harvest tank, a chromatography skid, a chromatography column, an ultrafiltration (UF) skid, a diafiltration (DF) skid, a UF/DF skid, a fill finish tank or an incubator.

In some embodiments, the Raman spectrum of the sample is compared to the Raman spectrum of a control sample.

In some embodiments, the spectral range of the sample is from about 100 to about 3600 cm⁻¹. In further embodiments, the spectral range of the sample is from about 200 to about 3600 cm⁻¹. In further embodiments, the spectral range of the sample is from about 425 to about 1800 cm⁻¹. In yet further embodiments, the spectral range of the sample is from about 2800 to about 3100 cm⁻¹.

In some embodiments, the spectral range of the control sample is from about 100 to about 3600 cm⁻¹. In further embodiments, the spectral range of the control sample is from about 200 to about 3600 cm⁻¹. In further embodiments, the spectral range of the control sample is from about 425 to about 1800 cm⁻¹. In yet further embodiments, the spectral range of the control sample is from about 2800 to about 3100 cm⁻¹

In some embodiments, the Raman spectrum of the sample is monitored over a period of time.

In some embodiments, the monitoring is automated.

In some embodiments, the monitoring is continuous.

In some embodiments, the monitoring is non-invasive and/or non-destructive.

In some embodiments, the test agent is a compound, a supplement, an antibiotic, a bacteriophage, or a combination thereof.

In some embodiments, the sample has been contacted with an antibody to the microbe.

In some embodiments, the sample is contacted with a fluorescent agent or a bioluminescent agent.

In some embodiments, the system further comprises a means for detecting fluorescence of the sample. In further embodiments, the means for detecting fluorescence is a fluorimeter.

In some embodiments, the system further comprises a means for detecting bioluminescence of the sample. In further embodiments, the means for detecting bioluminescence is a luminometer.

In some embodiments, the microbe includes but is not limited to Cutibacterium acnes, Staphylococcus aureus, Aspergillus brasiliensis, Candida albicans, Bacillus subtilis, Escherichia coli, Pseudomonas aeruginosa, Staphylococcus epidermidis, Streptococcus pyogenes, Micrococcus luteus, Burkholderia cepacia, Salmonella typhimurium, or Bacillus cereus. In some embodiments, the microbe is Cutibacterium acnes, Staphylococcus aureus, Aspergillus brasiliensis, Candida albicans, Bacillus subtilis, Escherichia coli, Pseudomonas aeruginosa, Staphylococcus epidermidis, Streptococcus pyogenes, Micrococcus luteus, Burkholderia cepacia, Salmonella typhimurium, or Bacillus cereus.

In some embodiments, the measuring of the Raman spectrum is automated.

In some embodiments, the Raman spectrum is subjected to data analysis.

In some embodiments, data analysis of the Raman spectrum comprises discriminant modeling performed using PCA-X, OPLS, KNN, OPLS-DA, PLS-DA, or combinations thereof.

In some embodiments, the method further comprises incubation with D2O for assessment of microbial viability. In further embodiments, a sample from a cell culture vessel is diverted to a flow cell chamber for infrared analysis. In some embodiments, the derivatization is performed prior to measuring the infrared spectrum of the sample.

In some embodiments, the sample comprises a eukaryotic cell.

In some embodiments, the eukaryotic cell produces a protein, an antibody, a duobody, a receptor, a chimeric antigen receptor, a glycoprotein, or a combination thereof.

In some embodiments, the eukaryotic cell is a mouse cell, a chinese hamster ovary (CHO) cell, or a human cell. In some embodiments, the cell is a T cell, or a B cell. In some embodiments, the mouse cell is a mouse Sp2/0 cell. In some embodiments, the cell is HEK293F. In some embodiments, the cell is PER.C6.

In some embodiments, the eukaryotic cell is a chimeric antigen receptor T cell (CAR-T cell).

In some embodiments, the sample is not centrifuged prior to measuring the Raman spectrum of the sample.

In some embodiments, the microbe is actively growing.

### DESCRIPTION OF THE FIGURES

FIG. 1 shows a visual representation of each spiking study with offline parallel plating.

FIG. 2 shows a typical spectrum (before and after pre-processing) during a bioreactor media only and cell culture processes with relevant wavenumber regions highlighted.

FIGS. 3A-3D illustrate principal component analysis (PCA) media-only model overview. FIG. 3A: Root mean square error of calibration (RMSEC) and root mean square error of cross-validation (RMSECV) v. principal component (PC). FIG. 3B: Q residuals v. sample. FIG. 3C: T2 v. sample. FIG. 3D: Scores.

FIGS. 4A-4E illustrate PCA cell culture only model overview. FIG. 4A: RMSEC and RMSECV v. PC. FIG. 4B: Q residuals v. sample. FIG. 4C: T2 v. sample. FIG. 4D: Scores. FIG. 4E: Scores.

FIGS. 5A-5H show an orthogonal partial least squares discriminant analysis (OPLS-DA) cell culture model overview. FIG. 5A: RMSEC and RMSECV v PC. FIG. 5B: T2 v sample. FIG. 5C: Q residuals v sample. FIG. 5D: Scores. FIG. 5E: Calibration error. FIG. 5F: Y Prediction plot. FIG. 5G: receiver operating characteristic (ROC) curve. FIG. 5H: Sensitivity v. Specificity.

FIG. 6A-6B illustrate k-nearest neighbors (KNN) cell culture model overview. FIG. 6A: Calibration error. FIG. 6B: Prediction plot.

FIGS. 7A-7B show the contamination trajectory predicted with the OPLS-DA cell culture model for uncontaminated manufacturing scale batch (FIG. 7A) and contaminated reduced scale batch (FIG. 7B) from a calibration test sample set (CTSS). Contamination was detected by plating at t=193 hrs for the small scale batch, indicated by the red star. The dashed line indicates predicted Y-values in the prediction set (YpredPS) limit of 0.35 to classify spectra as borderline while the solid line indicates YPredPS limit of 0.65 to classify spectra as contaminated. The data points for each sample are coded according to contaminated (black), noncontaminated (light gray), and ambiguous (dark gray).

FIG. 8 shows a table: Study Results Demonstrating the Raman Method Faster Time-to-Detection of Bioreactor Microbial Contamination Compared to Conventional Spread Plating.

FIGS. 9A-9F show a PLS-DA cell culture model overview for Stelara® (ustekinumab; Janssen) with the following microbial species tested: Staphylococcus epidermidis, Escherichia coli, Candida albicans, Bacillus cereus, Cutibacterium acnes, Bacillus subtilis, and Aspergillus brasiliensis. FIG. 9A: RMSECV v PC. FIG. 9B: T2 v sample. FIG. 9C: Distance to Model (DmodX) v sample. FIG. 9D: cumulative r-squared (R2Cum) and cumulative Q-square index (Q2Cum) v PC. FIG. 9E: Scores. FIG. 9F: Y Prediction plot. The data points for each sample are color coded according to contaminated (black) versus noncontaminated (grey).

FIGS. 10A-10C show a prediction of OPLS-DA contamination model on CTSS with ambiguous transition spectra for Stelara® (ustekinumab; Janssen) with the following microbial species tested: Staphylococcus epidermidis, Escherichia coli, Bacillus cereus, Bacillus subtilis, and control tests from reactor groupings from the remaining species in the calibration sample set (CSS). FIG. 10A: predicted values in the workset (Ypred) v sample. FIG. 10B: Y Prediction plot. The dashed line indicates YPredPS limit of 0.35 to classify spectra as borderline while the solid line indicates YPredPS limit of 0.65 to classify spectra as contaminated. FIG. 10C: ROC plot. The AUC is 0.716. The data points for each sample are color coded according to contaminated (black), noncontaminated (light grey), and ambiguous (dark grey).

FIGS. 11A-11C show prediction of OPLS-DA contamination model on CTSS without ambiguous transition spectra for Stelara® (ustekinumab; Janssen) with the following microbial species tested: Staphylococcus epidermidis, Escherichia coli, Bacillus cereus, Bacillus subtilis, and control tests from reactor groupings from the remaining species in the CSS. FIG. 11A: YPred v sample. FIG. 11B: Y Prediction plot. The dashed line indicates YPredPS limit of 0.35 to classify spectra as borderline while the solid line indicates YPredPS limit of 0.65 to classify spectra as contaminated. FIG. 11C: ROC plot. The AUC is 0.997. The data points for each sample are color coded according to contaminated (black) and noncontaminated (grey).

FIGS. 12A-12B shows batch trajectory prediction for Stelara® (ustekinumab; Janssen) uncontaminated reduced scale batch (FIG. 12A) and contaminated reduced scale batch from CTSS containing Bacillus cereus (FIG. 12B). Contamination was detected by plating at t=46.5 hrs for the small scale batch, indicated by the star. The dashed line indicates YPredPS limit of 0.35 to classify spectra as borderline while the solid line indicates YPredPS limit of 0.65 to classify spectra as contaminated. The data points for each sample are color coded according to contaminated (black), noncontaminated (light grey), and ambiguous (dark grey).

### DETAILED DESCRIPTION

While the general inventive concepts are susceptible of embodiment in many forms, there are shown in the drawings, and will be described herein in detail, specific embodiments thereof with the understanding that the present disclosure is to be considered an exemplification of the principles of the general inventive concepts. Accordingly, the general inventive concepts are not intended to be limited to the specific embodiments illustrated herein.

It is also to be understood that the terminology used herein is for the purpose of describing particular embodiments only, and is not intended to be limiting.

The articles “a” and “an” are used herein to refer to one or more than one (i.e., to at least one) of the grammatical object of the article. By way of example, “a cell” means one cell or more than one cell.

“About” as used herein when referring to a measurable value such as an amount, a temporal duration, and the like, is meant to encompass variations of ±5%, preferably ±1%, and still more preferably ±0.1% from the specified value, as such variations are appropriate to perform the disclosed methods.

As used herein the term “coherent light source” means a light source that emits light wave(s) having the same frequency, wavelength and in the same phase, or having a constant phase difference.

The terms “antibody” and “antibodies” as used herein are meant in a broad sense and include immunoglobulin molecules including polyclonal antibodies, monoclonal antibodies including murine, human, human-adapted, humanized and chimeric monoclonal antibodies, antibody fragments, bispecific or multispecific antibodies, dimeric, tetrameric or multimeric antibodies, and single chain antibodies.

Immunoglobulins can be assigned to five major classes, namely IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species can be assigned to one of two clearly distinct types, namely kappa (κ) and lambda (λ), based on the amino acid sequences of their constant domains.

The term “antibody fragments” refers to a portion of an immunoglobulin molecule that retains the heavy chain and/or the light chain antigen binding site, such as heavy chain complementarity determining regions (HCDR) 1, 2 and 3, light chain complementarity determining regions (LCDR) 1, 2 and 3, a heavy chain variable region (VH), or a light chain variable region (VL). Antibody fragments include a Fab fragment, a monovalent fragment consisting of the VL, VH, CL and CH1 domains; a F(ab)₂ fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a domain antibody (dAb) fragment, which consists of a VH domain. VH and VL domains can be engineered and linked together via a synthetic linker to form various types of single chain antibody designs where the VH/VL domains pair intramolecularly, or intermolecularly in those cases when the VH and VL domains are expressed by separate single chain antibody constructs, to form a monovalent antigen binding site, such as single chain Fv (scFv) or diabody; described for example in PCT Intl. Publ. Nos. WO1998/44001, WO1988/01649, WO1994/13804, and WO1992/01047. These antibody fragments are obtained using well known techniques known to those of skill in the art, and the fragments are screened for utility in the same manner as are full length antibodies.

The phrase “isolated antibody” refers to an antibody or antibody fragment that is substantially free of other antibodies having different antigenic specificities (e.g., an isolated antibody specifically binding CD38 is substantially free of antibodies that specifically bind antigens other than human CD38). An isolated antibody that specifically binds CD38, however, can have cross-reactivity to other antigens, such as orthologs of human CD38, such as Macaca fascicularis (cynomolgus monkey) CD38. Moreover, an isolated antibody may be substantially free of other cellular material and/or chemicals.

“Humanized antibody” refers to an antibody in which the antigen binding sites are derived from non-human species and the variable region frameworks are derived from human immunoglobulin sequences. Humanized antibodies may include substitutions in the framework regions so that the framework may not be an exact copy of expressed human immunoglobulin or germline gene sequences.

“Human antibody” refers to an antibody having heavy and light chain variable regions in which both the framework and the antigen binding sites are derived from sequences of human origin. If the antibody contains a constant region, the constant region also is derived from sequences of human origin. A human antibody comprises heavy or light chain variable regions that are “derived from” sequences of human origin wherein the variable regions of the antibody are obtained from a system that uses human germline immunoglobulin or rearranged immunoglobulin genes. Such systems include human immunoglobulin gene libraries displayed on phage, and transgenic non-human animals such as mice carrying human immunoglobulin loci as described herein. A human antibody may also contain amino acid differences when compared to the human germline or rearranged immunoglobulin sequences due to for example naturally occurring somatic mutations or intentional introduction of substitutions in the framework or antigen binding sites. Typically, a human antibody is at least about 80%, 81%, 82%, 83%, 84%, 85%, 86%, 87%, 88%, 89%, 90%, 91%, 92%, 93%, 94%, 95%, 96%, 97%, 98%, 99% or 100% identical in amino acid sequence to an amino acid sequence encoded by a human germline or rearranged immunoglobulin gene.

Isolated humanized antibodies may be synthetic. Human antibodies, while derived from human immunoglobulin sequences, may be generated using systems such as phage display incorporating synthetic CDRs and/or synthetic frameworks, or can be subjected to in vitro mutagenesis to improve antibody properties, resulting in antibodies that do not naturally exist within the human antibody germline repertoire in vivo.

The term “recombinant antibody” as used herein, includes all antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the antibody, antibodies isolated from a recombinant, combinatorial antibody library, and antibodies prepared, expressed, created or isolated by any other means that involve splicing of human immunoglobulin gene sequences to other DNA sequences, sequences, or antibodies that are generated in vitro using Fab arm exchange such as bispecific antibodies.

The term “monoclonal antibody” as used herein refers to a preparation of antibody molecules of single molecular composition. A monoclonal antibody composition displays a single binding specificity and affinity for a particular epitope, or in a case of a bispecific monoclonal antibody, a dual binding specificity to two distinct epitopes.

The term “epitope” as used herein means a portion of an antigen to which an antibody specifically binds. Epitopes usually consist of chemically active (such as polar, non-polar or hydrophobic) surface groupings of moieties such as amino acids or polysaccharide side chains and can have specific three-dimensional structural characteristics, as well as specific charge characteristics. An epitope can be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule.

The term “chimeric antigen receptor” or “CAR” as used herein means a synthetic or recombinant receptor comprising an antigen specific domain, a costimulatory domain and an intracellular signaling domain. In some embodiments, the CAR further comprises an extracellular hinge or spacer region, a transmembrane domain, or combinations thereof. In some embodiments, the antigen specific domain is an scFv.

The term “chimeric antigen receptor T cell” or “CAR-T” as used herein means a T cell expressing a CAR.

As used herein, the term “bioreactor” generally refers to a device that supports a biologically active process, such as the culture of cells. Exemplary bioreactors include stainless steel stirred bioreactors, air-lift reactors, and disposable bioreactors.

As used herein, the term “in-line” generally means that the measuring or determining is performed in real time by a probe placed in a container (for example, in a bioreactor) and collection of a sample is not required.

In some embodiments of any of the compositions or methods described herein, a range is intended to comprise every integer or fraction or value within the range.

Embodiments described herein as “comprising” one or more features may also be considered as disclosure of the corresponding embodiments “consisting of” and/or “consisting essentially of” such features.

To date the use of an in-line Raman sensor as an automated, non-invasive, non-destructive detection of actively growing microbial contamination directly in mammalian cell bioreactors has not been proposed. Provided herein are methods that result in eliminating the process interventions currently needed for collecting bioreactor samples for conventional spread plate analysis, which has an added benefit of reducing daily risk of cross contamination in bioreactors. In addition, the versatility of the Raman sensor to provide other process monitoring test (PMT) and IPC measurements sets the stage for elimination of bioreactor sampling altogether. During early Raman method development internally, it was observed that when a non-intentional microbiological contamination occurred in the bioreactor, the Raman probe sensed a change in the cell culture through both a change in the metabolic profile (e.g. glucose consumption and lactate generation) as well as a multivariate models that serve as the biological process fingerprint. These models were useful to identify when a process deviated from expected nominal conditions. In this work, Raman multivariate analysis capability was further explored to determine whether it also had the specificity to identify different types of microbial contamination (i.e. fast vs. slow, and aerobic vs. anaerobic) and distinguish it from other process faults (under or over feed, pH, or gas control, etc.). The methods developed by the inventors of the present disclosure demonstrated equivalence or superiority to the compendial methods with regards to time to detection, detected presence of all organisms tested, and can be validated using guidance documents for alternatives to compendial methods.²³⁻²⁴

Raman spectroscopy may include Surface Enhanced Raman Spectroscopy (SERS), Spatially Offset Raman spectroscopy (SORS), transmission Raman spectroscopy, and/or resonance Raman spectroscopy.

### Methods

### Methods for Detecting a Microbe

Provided is a method for detecting a microbe in a sample comprising: measuring a Raman spectrum of the sample.

In some embodiments, the sample is exposed to a coherent light source prior to measuring the Raman spectrum of the sample.

### Methods for Continuous Monitoring

Provided is a method for continuous monitoring for microbial presence in a sample comprising: measuring a Raman spectrum of the sample.

In some embodiments, the Raman spectrum of the sample is measured at set intervals. In some embodiments, the Raman spectrum is measured at random intervals. In some embodiments, the Raman spectrum measurement is uninterrupted.

In some embodiments, the Raman spectrum of the sample is monitored over a period of time. In further embodiments, the Raman spectrum of the sample is monitored at set intervals, over a period of time. In yet further embodiments, the Raman spectrum of the sample is monitored at random intervals, over a period of time. In yet further embodiments, the Raman spectrum of the sample is monitored continuously, over a period of time. The period of time may be from about 0.1 to about 1000 hours, from about 0.1 to about 100 hours, from about 0.1 to about 10 hours, from about 0.1 to about 1 hour, or from about 0.1 to about 0.2 hours.

In some embodiments, the monitoring is automated.

In some embodiments, the monitoring is non-invasive and/or non-destructive.

In some embodiments, the sample is exposed to a coherent light source prior to measuring the Raman spectrum of the sample.

### Methods for Assaying a Test Agent

Provided is a method for assaying a test agent comprising: adding a test agent to a sample comprising a microbe; and measuring a Raman spectrum of the sample.

In some embodiments, the sample is exposed to a coherent light source prior to measuring the Raman spectrum of the sample.

### Raman Spectrometer

In some embodiments, the Raman spectrum is measured using a Raman spectrometer. The Raman spectrometer may be commercially available, for example a Kaiser Optical Systems RXN2 (Endress Hauser, Reinach Switzerland). In some embodiments, the Raman spectrometer is built from individual components (for example, laser, spectrometer, detector) commercially available.

In some embodiments, the Raman spectrometer comprises a laser. In further embodiments, the laser is a multimode diode laser.

In some embodiments, the laser wavelength is from 300 nm to 1200 nm, from 350 nm to 1100 nm, from 400 nm to 1100 nm, from 400 nm to 1064 nm, from 450 nm to 1064 nm, from 500 nm to 1064 nm, from 550 nm to 1064 nm, from 600 nm to 1064 nm, from 650 nm to 1064 nm, from 700 nm to 1064 nm, from 450 nm to 1100 nm, or from 500 nm to 1100 nm. In any embodiment, the light source is a narrow bandwidth laser with a wavelength of about 800 nm, about 785 nm, about 750 nm, about 725 nm, about 700 nm, about 675 nm, about 650 nm, about 625 nm, about 600 nm, about 575 nm, about 550 nm, about 532 nm, about 525 nm, or about 500 nm.

In some embodiments, the laser wavelength is 785 nm.

### Growth Conditions for the Sample

In some embodiments, the sample is in a bioreactor, a harvest tank, a chromatography skid, a chromatography column, an ultrafiltration (UF) skid, a diafiltration (DF) skid, a UF/DF skid, a fill finish tank or an incubator.

The bioreactor can have any suitable volume that allows for the cultivation and propagation of biological cells capable of producing therapeutic proteins (such as Stelara® (ustekinumab; Janssen)). For example, the volume of the bioreactor can be about 0.5 liters (L) to about 25,000 L. In some embodiments, the volume of the bioreactor can be less than or equal to about 250 L. In some embodiments, the volume of the bioreactor can be about 0.5 liters (L) to about 250 L. In some embodiments, the volume of the bioreactor can be less than or equal to about 50 L. In some embodiments, the volume of the bioreactor can be about 1 L to about 50 L. In some embodiments, the volume of the bioreactor can be less than or equal to about 25 L. In some embodiments, the volume of the bioreactor can be about 1 L to about 25 L. In some embodiments, the volume of the bioreactor can be less than or equal to about 250 L. In some embodiments, the volume of the bioreactor can be less than or equal to about 100 L. In some embodiments, the volume of the bioreactor can be less than or equal to about 50 L. In some embodiments, the volume of the bioreactor can be less than or equal to about 25 L. In some embodiments, the volume of the bioreactor can be less than or equal to about 10 L. In some embodiments, the volume of the bioreactor can be less than or equal to about 5 L. In some embodiments, the volume of the bioreactor can be less than or equal to about 1 L. In some embodiments, the volume of the bioreactor can be about 1 L. In some embodiments, the volume of the bioreactor can be about 2 L. In some embodiments, the volume of the bioreactor can be about 5 L. In some embodiments, the volume of the bioreactor can be equal to or about 1,000 L. In some embodiments, the volume of the bioreactor can be about 1,000 L to about 25,000 L. In some embodiments, the volume of the bioreactor can be about 10,000 L to about 25,000 L. In some embodiments, the volume of the bioreactor can be about 1,000 L. In some embodiments, the volume of the bioreactor can be about 2,000 L. In some embodiments, the volume of the bioreactor can be about 5,000 L. In some embodiments, the volume of the bioreactor can be about 10,000 L. In some embodiments, the volume of the bioreactor can be about 15,000 L. In some embodiments, the volume of the bioreactor can be about 25,000 L.

In some embodiments, the sample is at a temperature from about 2° C. to about 40° C. In some embodiments, the sample is at a temperature from about 20° C. to about 40° C. In some embodiments, the preferred temperature is from about 25° C. to about 37° C. In further embodiments, the sample is at a temperature from about 2° C. to about 20° C. In yet further embodiments, the sample is at a temperature from about 2° C. to about 10° C.

In some embodiments, the sample is grown for about 0.5 to about 14 days prior to measuring the Raman spectrum of the sample. In some embodiments, the sample is grown for about 0.5 to about 7 days prior to measuring the Raman spectrum of the sample. In further embodiments, the sample is grown for about 0.5, about 1, about 2, about 3, about 4, about 5, about 6, or about 7 days prior to measuring the Raman spectrum of the sample.

In some embodiments, the Raman spectrum of the sample is compared to the Raman spectrum of a control sample.

In some embodiments, the spectral range of the sample is from about 100 to about 3600 cm⁻¹. In further embodiments, the spectral range of the sample is from about 200 to about 3600 cm⁻¹. In further embodiments, the spectral range of the sample is from about 425 to about 1800 cm⁻¹. In yet further embodiments, the spectral range of the sample is from about 2800 to about 3100 cm⁻¹.

In some embodiments, the spectral range of the control sample is from about 100 to about 3600 cm⁻¹. In further embodiments, the spectral range of the control sample is from about 200 to about 3600 cm⁻¹. In further embodiments, the spectral range of the control sample is from about 425 to about 1800 cm⁻¹. In yet further embodiments, the spectral range of the control sample is from about 2800 to about 3100 cm⁻¹.

In some embodiments, the Raman spectrum of the sample is monitored over a period of time. In further embodiments, the Raman spectrum of the sample is monitored at set intervals, over a period of time. In yet further embodiments, the Raman spectrum of the sample is monitored at random intervals, over a period of time. In yet further embodiments, the Raman spectrum of the sample is monitored continuously, over a period of time. The period of time may be from about 0.1 to about 1000 hours, from about 0.1 to about 100 hours, from about 0.1 to about 10 hours, from about 0.1 to about 1 hour, or from about 0.1 to about 0.2 hours.

In some embodiments, the monitoring is automated.

In some embodiments, the monitoring is continuous.

In some embodiments, the monitoring is non-invasive and/or non-destructive.

In some embodiments, the sample has been contacted with an antibody to the microbe.

In some embodiments, the sample is contacted with a fluorescent agent or a bioluminescent agent.

In some embodiments, the method further comprises detecting fluorescence of the sample. In further embodiments, the means for detecting fluorescence is a fluorimeter.

In some embodiments, the method further comprises detecting bioluminescence of the sample. In further embodiments, the means for detecting bioluminescence is a luminometer.

In some embodiments, the microbe includes but is not limited to Cutibacterium acnes, Staphylococcus aureus, Aspergillus brasiliensis, Candida albicans, Bacillus subtilis, Escherichia coli, Pseudomonas aeruginosa, Staphylococcus epidermidis, Streptococcus pyogenes, Micrococcus luteus, Burkholderia cepacia, Salmonella typhimurium, or Bacillus cereus. In some embodiments, the microbe is Cutibacterium acnes, Staphylococcus aureus, Aspergillus brasiliensis, Candida albicans, Bacillus subtilis, Escherichia coli, Pseudomonas aeruginosa, Staphylococcus epidermidis, Streptococcus pyogenes, Micrococcus luteus, Burkholderia cepacia, Salmonella typhimurium, or Bacillus cereus.

In some embodiments, the measuring of the Raman spectrum is automated. In some embodiments, the automation is controlled by a central processor, for example a computer. In further embodiments, the automation is robotic.

### Data Analysis

In some embodiments, the Raman spectrum is subjected to data analysis.

In some embodiments, data analysis of the Raman spectrum comprises discriminant modeling performed using principal component analysis-X (PCA-X), orthogonal partial least squares (OPLS), k-nearest neighbors (KNN), orthogonal partial least squares discriminant analysis (OPLS-DA), partial least squares discriminant analysis (PLS-DA), or combinations thereof. In some embodiments, data analysis comprises a model for a microbe including but not limited to Cutibacterium acnes, Staphylococcus aureus, Aspergillus brasiliensis, Candida albicans, Bacillus subtilis, Escherichia coli, Pseudomonas aeruginosa, Staphylococcus epidermidis, Streptococcus pyogenes, Micrococcus luteus, Burkholderia cepacia, Salmonella typhimurium, or Bacillus cereus.

In some embodiments, data analysis comprises generating one or more models based on the obtained Raman spectrum data that correlate levels of microbe with the obtained Raman spectrum data. In some embodiments, the one or more models are regression models.

In some embodiments, data analysis is executed by one or more computing devices.

In some embodiments, the Raman spectrometer is connected to one or more data processors and memory into which instructions can be loaded and executed by the data processors. In some embodiments, the data processors communicate over a network with one or more computing systems (e.g., servers, personal computers, tablets, IoT devices, mobile phones, dedicated control units, etc.) which can execute various algorithms or data analysis as described elsewhere herein. The computing systems can also act to change one or more operating parameters associated with the bioreactor. In some cases, the bioreactor can also have network connectivity such that it can communicate with one or more remote computing systems which, in turn, can cause one or more operating parameters of the bioreactor to change.

Derivatization with D2O

In some embodiments, the method further comprises incubation with D2O for assessment of microbial viability. In further embodiments, a sample from a cell culture vessel is diverted to a flow cell chamber for Raman analysis. In some embodiments, the derivatization is performed prior to measuring the Raman spectrum of the sample.

### Cells

In some embodiments, the sample comprises a eukaryotic cell. In further embodiments, the eukaryotic cell produces a therapeutic product. The therapeutic product may be released into the cell media, where it may be collected. The methods provided herein allow for detection of contamination.

In some embodiments, the eukaryotic cell produces a protein, an antibody or fragment thereof, a duobody, a receptor, a chimeric antigen receptor, a glycoprotein, a viral vector, or a combination thereof.

In some embodiments, the eukaryotic cell is a mouse cell, a chinese hamster ovary (CHO) cell, or a human cell. In some embodiments, the cell is a T cell, or a B cell. In some embodiments, the mouse cell is a mouse Sp2/0 cell. In some embodiments, the cell is HEK293F. In some embodiments, the cell is PER.C6.

In some embodiments, the eukaryotic cell is a chimeric antigen receptor T cell (CAR-T cell).

In some embodiments, the sample is not centrifuged prior to measuring the Raman spectrum of the sample. In some embodiments, the sample is not removed from the bioreactor, harvest tank, chromatography skid, chromatography column, ultrafiltration (UF) skid, diafiltration (DF) skid, UF/DF skid, fill finish tank or incubator prior to measuring the Raman spectrum of the sample.

In some embodiments, the microbe is actively growing.

Advantages of the methods provided herein include but are not limited to risk reduction of encountering false positive process samples due to QC microbiology lab cross contamination, cost reduction, and combinations thereof.

### Systems

### Systems for Detecting a Microbe

Provided is a system for detecting a microbe in a sample comprising: a means for measuring a Raman spectrum of the sample.

In some embodiments, the system further comprises a coherent light source.

### Systems for Continuous Monitoring

Provided is a system for continuous monitoring for microbial presence in a sample comprising: a means for measuring a Raman spectrum of the sample.

In some embodiments, the system further comprises a coherent light source.

In some embodiments, the Raman spectrum of the sample is measured at set intervals. In some embodiments, the Raman spectrum is measured at random intervals. In some embodiments, the Raman spectrum measurement is uninterrupted.

In some embodiments, the Raman spectrum of the sample is monitored over a period of time. In further embodiments, the Raman spectrum of the sample is monitored at set intervals, over a period of time. In yet further embodiments, the Raman spectrum of the sample is monitored at random intervals, over a period of time. In yet further embodiments, the Raman spectrum of the sample is monitored continuously, over a period of time. The period of time may be from about 0.1 to about 1000 hours, from about 0.1 to about 100 hours, from about 0.1 to about 10 hours, from about 0.1 to about 1 hour, or from about 0.1 to about 0.2 hours.

In some embodiments, the monitoring is non-invasive and/or non-destructive.

In some embodiments, the sample is exposed to a coherent light source prior to measuring the Raman spectrum of the sample.

### Systems for Assaying a Test Agent

Provided is a system for assaying a test agent comprising: a means for measuring a Raman spectrum of a sample to which a test agent has been added.

In some embodiments, the system further comprises a coherent light source.

### Raman Spectrometer

In some embodiments, the Raman spectrum is measured using a Raman spectrometer. The Raman spectrometer may be commercially available, for example a Kaiser Optical Systems RXN2 (Endress Hauser, Reinach Switzerland). In some embodiments, the Raman spectrometer is built from individual components (for example, laser, spectrometer, detector) commercially available.

In some embodiments, the Raman spectrometer comprises a laser. In further embodiments, the laser is a multimode diode laser.

In some embodiments, the laser wavelength is from 300 nm to 1200 nm, from 350 nm to 1100 nm, from 400 nm to 1100 nm, from 400 nm to 1064 nm, from 450 nm to 1064 nm, from 500 nm to 1064 nm, from 550 nm to 1064 nm, from 600 nm to 1064 nm, from 650 nm to 1064 nm, from 700 nm to 1064 nm, from 450 nm to 1100 nm, or from 500 nm to 1100 nm. In any embodiment, the light source is a narrow bandwidth laser with a wavelength of about 800 nm, about 785 nm, about 750 nm, about 725 nm, about 700 nm, about 675 nm, about 650 nm, about 625 nm, about 600 nm, about 575 nm, about 550 nm, about 532 nm, about 525 nm, or about 500 nm.

In some embodiments, the laser wavelength is 785 nm.

### Growth Conditions for the Sample

In some embodiments, the sample is in a bioreactor, a harvest tank, a chromatography skid, a chromatography column, an ultrafiltration (UF) skid, a diafiltration (DF) skid, a UF/DF skid, a fill finish tank or an incubator.

The bioreactor can have any suitable volume that allows for the cultivation and propagation of biological cells capable of producing therapeutic proteins (such as Stelara® (ustekinumab; Janssen)). For example, the volume of the bioreactor can be about 0.5 liters (L) to about 25,000 L. In some embodiments, the volume of the bioreactor can be less than or equal to about 250 L. In some embodiments, the volume of the bioreactor can be about 0.5 liters (L) to about 250 L. In some embodiments, the volume of the bioreactor can be less than or equal to about 50 L. In some embodiments, the volume of the bioreactor can be about 1 L to about 50 L. In some embodiments, the volume of the bioreactor can be less than or equal to about 25 L. In some embodiments, the volume of the bioreactor can be about 1 L to about 25 L. In some embodiments, the volume of the bioreactor can be less than or equal to about 250 L. In some embodiments, the volume of the bioreactor can be less than or equal to about 100 L. In some embodiments, the volume of the bioreactor can be less than or equal to about 50 L. In some embodiments, the volume of the bioreactor can be less than or equal to about 25 L. In some embodiments, the volume of the bioreactor can be less than or equal to about 10 L. In some embodiments, the volume of the bioreactor can be less than or equal to about 5 L. In some embodiments, the volume of the bioreactor can be less than or equal to about 1 L. In some embodiments, the volume of the bioreactor can be about 1 L. In some embodiments, the volume of the bioreactor can be about 2 L. In some embodiments, the volume of the bioreactor can be about 5 L. In some embodiments, the volume of the bioreactor can be equal to or about 1,000 L. In some embodiments, the volume of the bioreactor can be about 1,000 L to about 25,000 L. In some embodiments, the volume of the bioreactor can be about 10,000 L to about 25,000 L. In some embodiments, the volume of the bioreactor can be about 1,000 L. In some embodiments, the volume of the bioreactor can be about 2,000 L. In some embodiments, the volume of the bioreactor can be about 5,000 L. In some embodiments, the volume of the bioreactor can be about 10,000 L. In some embodiments, the volume of the bioreactor can be about 15,000 L. In some embodiments, the volume of the bioreactor can be about 25,000 L.

In some embodiments, the sample is at a temperature from about 2° C. to about 40° C. In some embodiments, the sample is at a temperature from about 20° C. to about 40° C. In some embodiments, the preferred temperature is from about 25° C. to about 37° C. In further embodiments, the sample is at a temperature from about 2° C. to about 20° C. In yet further embodiments, the sample is at a temperature from about 2° C. to about 10° C.

In some embodiments, the sample is grown for about 0.5 to about 14 days prior to measuring the Raman spectrum of the sample. In some embodiments, the sample is grown for about 0.5 to about 7 days prior to measuring the Raman spectrum of the sample. In further embodiments, the sample is grown for about 0.5, about 1, about 2, about 3, about 4, about 5, about 6, or about 7 days prior to measuring the Raman spectrum of the sample.

In some embodiments, the Raman spectrum of the sample is compared to the Raman spectrum of a control sample.

In some embodiments, the spectral range of the sample is from about 100 to about 3600 cm⁻¹. In further embodiments, the spectral range of the sample is from about 200 to about 3600 cm⁻¹. In further embodiments, the spectral range of the sample is from about 425 to about 1800 cm⁻¹. In yet further embodiments, the spectral range of the sample is from about 2800 to about 3100 cm⁻¹.

In some embodiments, the spectral range of the control sample is from about 100 to about 3600 cm⁻¹. In further embodiments, the spectral range of the control sample is from about 200 to about 3600 cm⁻¹. In further embodiments, the spectral range of the control sample is from about 425 to about 1800 cm⁻¹. In yet further embodiments, the spectral range of the control sample is from about 2800 to about 3100 cm⁻¹.

In some embodiments, the Raman spectrum of the sample is monitored over a period of time. In further embodiments, the Raman spectrum of the sample is monitored at set intervals, over a period of time. In yet further embodiments, the Raman spectrum of the sample is monitored at random intervals, over a period of time. In yet further embodiments, the Raman spectrum of the sample is monitored continuously, over a period of time. The period of time may be from about 0.1 to about 1000 hours, from about 0.1 to about 100 hours, from about 0.1 to about 10 hours, from about 0.1 to about 1 hour, or from about 0.1 to about 0.2 hours.

In some embodiments, the monitoring is automated.

In some embodiments, the monitoring is continuous.

In some embodiments, the monitoring is non-invasive and/or non-destructive.

In some embodiments, the sample has been contacted with an antibody to the microbe.

In some embodiments, the sample is contacted with a fluorescent agent or a bioluminescent agent.

In some embodiments, the system further comprises a means for detecting fluorescence of the sample. In further embodiments, the means for detecting fluorescence is a fluorimeter.

In some embodiments, the system further comprises a means for detecting bioluminescence of the sample. In further embodiments, the means for detecting bioluminescence is a luminometer.

In some embodiments, the microbe includes but is not limited to Cutibacterium acnes, Staphylococcus aureus, Aspergillus brasiliensis, Candida albicans, Bacillus subtilis, Escherichia coli, Pseudomonas aeruginosa, Staphylococcus epidermidis, Streptococcus pyogenes, Micrococcus luteus, Burkholderia cepacia, Salmonella typhimurium, or Bacillus cereus. In some embodiments, the microbe is Cutibacterium acnes, Staphylococcus aureus, Aspergillus brasiliensis, Candida albicans, Bacillus subtilis, Escherichia coli, Pseudomonas aeruginosa, Staphylococcus epidermidis, Streptococcus pyogenes, Micrococcus luteus, Burkholderia cepacia, Salmonella typhimurium, or Bacillus cereus.

In some embodiments, the measuring of the Raman spectrum is automated. In some embodiments, the automation is controlled by a central processor, for example a computer. In further embodiments, the automation is robotic.

### Data Analysis

In some embodiments, the Raman spectrum is subjected to data analysis.

In some embodiments, data analysis of the Raman spectrum comprises discriminant modeling performed using principal component analysis-X (PCA-X), orthogonal partial least squares (OPLS), k-nearest neighbors (KNN), orthogonal partial least squares discriminant analysis (OPLS-DA), partial least squares discriminant analysis (PLS-DA), or combinations thereof. In some embodiments, data analysis comprises a model for a microbe including but not limited to Cutibacterium acnes, Staphylococcus aureus, Aspergillus brasiliensis, Candida albicans, Bacillus subtilis, Escherichia coli, Pseudomonas aeruginosa, Staphylococcus epidermidis, Streptococcus pyogenes, Micrococcus luteus, Burkholderia cepacia, Salmonella typhimurium, or Bacillus cereus.

In some embodiments, data analysis comprises generating one or more models based on the obtained Raman spectrum data that correlate levels of microbe with the obtained Raman spectrum data. In some embodiments, the one or more models are regression models.

In some embodiments, data analysis is executed by one or more computing devices.

In some embodiments, the Raman spectrometer is connected to one or more data processors and memory into which instructions can be loaded and executed by the data processors. In some embodiments, the data processors communicate over a network with one or more computing systems (e.g., servers, personal computers, tablets, IoT devices, mobile phones, dedicated control units, etc.) which can execute various algorithms or data analysis as described elsewhere herein. The computing systems can also act to change one or more operating parameters associated with the bioreactor. In some cases, the bioreactor can also have network connectivity such that it can communicate with one or more remote computing systems which, in turn, can cause one or more operating parameters of the bioreactor to change.

Derivatization with D2O

In some embodiments, the method further comprises incubation with D2O for assessment of microbial viability. In further embodiments, a sample from a cell culture vessel is diverted to a flow cell chamber for Raman analysis. In some embodiments, the derivatization is performed prior to measuring the Raman spectrum of the sample.

### Cells

In some embodiments, the sample comprises a eukaryotic cell. In further embodiments, the eukaryotic cell produces a therapeutic product. The therapeutic product may be released into the cell media, where it may be collected. The systems provided herein allow for detection of contamination.

In some embodiments, the eukaryotic cell produces a protein, an antibody or fragment thereof, a duobody, a receptor, a chimeric antigen receptor, a glycoprotein, a viral vector, or a combination thereof.

In some embodiments, the eukaryotic cell is a mouse cell, a chinese hamster ovary (CHO) cell, or a human cell. In some embodiments, the cell is a T cell, or a B cell. In some embodiments, the mouse cell is a mouse Sp2/0 cell. In some embodiments, the cell is HEK293F. In some embodiments, the cell is PER.C6.

In some embodiments, the eukaryotic cell is a chimeric antigen receptor T cell (CAR-T cell).

In some embodiments, the sample is not centrifuged prior to measuring the Raman spectrum of the sample. In some embodiments, the sample is not removed from the bioreactor, harvest tank, chromatography skid, chromatography column, ultrafiltration (UF) skid, diafiltration (DF) skid, UF/DF skid, fill finish tank or incubator prior to measuring the Raman spectrum of the sample.

In some embodiments, the microbe is actively growing.

Advantages of the systems provided herein include but are not limited to risk reduction of encountering false positive process samples due to QC microbiology lab cross contamination, cost reduction, and combinations thereof.

### EXAMPLES

### Example 1: Spiking Studies

### Materials and Methods

### Test Microorganism Culture Preparation and Plating Method

Test microorganisms (Biomerieux, USA, Bioball or equivalent (Remel Quanti-cult plus)) were resuspended according to manufacturers' instructions. A target suspension of ≤100 CFU per 0.1 mL were prepared to perform the designated inoculation studies.

The target colony forming units (CFUs) were confirmed by triplicate spread plate 0.1 mL aliquots of each test microorganism suspension onto individual pre-poured TSA plates in triplicate. The plates were incubated aerobically or anaerobically as designated for 1-3 days at 30-35° C. to determine the delivery counts spiked into each bioreactor.

Bioreactor samples were collected at least daily unless noted otherwise and spread plating (0.5 mL) performed in triplicate using a 1 μL inoculating loop or equivalent. A PBS only spread plate was used as the negative control. Samples were incubated at 35-37° C. and inspected daily to measure CFUs.

### Bioreactor Operations

The reduced scale bioreactors were either 1.5 or 3.0 L scale. Each bioreactor was equipped with pH, dissolved oxygen (DO), and Raman probes. The temperature was controlled at 36.5° C. The DO setpoint was 40%. There was no pH control. Proprietary basal media specific to a monoclonal antibody process was used in all studies. A mouse Sp2/0 cell line was used for cell culture reactors.

Media-only bioreactors were spiked at a low and high CFU target (5, 50 CFUs) after the temperature and DO were stabilized for a minimum of 6 hours. Once stabilized, the reactors were inoculated at 0.1 mL of the test microorganism suspension via a welded inoculation bottle or bag (suspension was diluted in 10 mL media) into each designated bioreactor and time of spiking recorded.

Cell culture bioreactors were inoculated with the mouse cell line at a target 0.3-0.5×10⁶ cells/mL. The cell culture was allowed to run for at least 24 hrs. prior to spiking microorganisms. The bioreactors were then inoculated with the test microorganism suspension and the time of spiking recorded. A low CFU target was applied to three of the organisms while two organisms were spiked at higher levels to achieve necessary growth.

### Raman Spectroscopy

Raman data was collected with a Kaiser Optical Systems RXN2 (Endress Hauser) equipped with the RunTime HMI operating system (version 5), 785 nm excitation laser, and a CCD camera maintained at 40° C. A fiber optic cable containing excitation and collection fibers were attached to bioreactors. Acquisition parameters were 10 s and 75 accumulations for a total collection time of 12.5 min per scan. Each Raman spectrum contained 100-3425 cm⁻¹ spectral region.

### Data Analysis

Spectra were preprocessed using derivatives, normalization, and wavelength selection (425-1800, 2800-3100 cm⁻¹). Wavelength selection involved removing peaks due to the optical window material and regions with no Raman information, as documented from instrument vendor. Data were divided between the calibration sample set (CSS) and calibration test set (CTS). Only known non-contaminated and contaminated spectra were used to develop the model with the CSS. Discriminant modeling was performed using PCA-X, OPLS, and KNNs using SIMCA Version 15.0, SIMCA Version 14.1, or Eigenvector PLS_Toolbox Version 8.9.2. Data analysis comprised of models for microbes tested.

For Eigenvector (Eigenvector Research Incorporated, Manson, WA) built models, filtering and despiking, extended multiplicative signal correction (EMSC) and Mean centering were used. For SIMCA, first derivative, 31 point smoothing and standard normal variate (SNV) was used.

The Raman method and offline plating were compared by a Nonparametric Wilcoxon test.

### Experimental Design

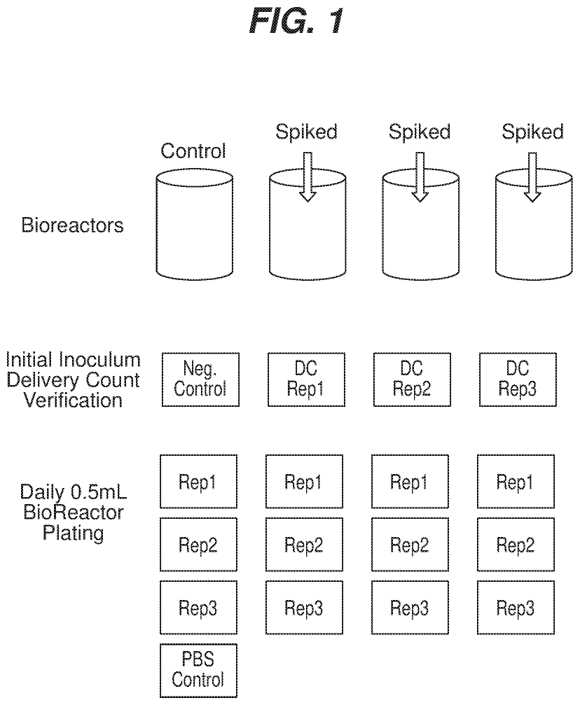

A study is defined as the test microorganism and spiking target. See FIG. 1. A set of four reduced-scale bioreactors were intentionally spiked with different organisms representing slow and fast growing as well as anaerobic and aerobic according to Tables 1-2. One reactor served as the control and was not spiked with a microorganism. The remaining three bioreactors were spiked. Daily triplicate spread plating of bioreactor samples, daily visual inspection for turbidity, and process parameters were documented. The organisms were selected based on compendial methods. One set of experiments focused on media-only bioreactors while the second set were cell-culture bioreactors running the process to simulate the first few days of a perfusion based process. For media-only reactors a low and high CFU target was used while the cell culture reactors used low CFU targets to establish the lower limits of detection.

TABLE 1
List of Organisms

| | | Approximate | |
|---|---|---|---|
| | | Empirically | |
| | | Derived | |
| Designated Test | | Doubling | |
| Microorganisms | Description | Time (hrs.) | Respiration Type |
| Candida albicans | Biomerieux BIOBALL Strain NCPF | 1.37 | Aerobic |
| | 3179 or equivalent | | |
| Escherichia coli | Biomerieux BIOBALL Strain NCTC | 0.28 | Aerobic |
| | 12923 or equivalent | | |
| Staphylococcus | Biomerieux BIOBALL Strain NCTC | 0.45 | Aerobic |
| epidermidis | 6513 or equivalent | | |
| Bacillus cereus | Biomerieux BIOBALL Strain NCTC | 0.30 | Anaerobic |
| | 7464 or equivalent or Janssen | | |
| | Environmental Isolate | | |
| Bacillus subtilis | Biomerieux BIOBALL Strain NCTC | 2.00 | Aerobic |
| subspecies | 10400 or equivalent | | |
| spizizenli | | | |
| Aspergillus | Biomerieux BIOBALL Strain NCPF | 1.50 | Aerobic |
| brasiliensis | 2275 or equivalent | | |
| Cutibacterium | Biomerieux BIOBALL Strain DSM | 5.00 | Anaerobic |
| acnes | 1897 or equivalent or Janssen | | |
| | Environmental Isolate | | |

TABLE 2
Media-only Bioreactor Designs

| | Bioreactor Spiking Inoculum Target Population Counts (colony-forming | |
|---|---|---|
| | units [CFU]) | |
| Test Microorganisms | ^{~} 50 CFU | ^{~} 15 CFU |

| | |
|---|---|
| Aspergillus brasiliensis | 1) Bioreactors spiked with designated microorganisms at |
| Bacillus cereus | two different target population counts |
| Bacillus subtilis | 2) Daily triplicate 1 mL spread plating of bioreactor |
| Candida albicans | samples performed in parallel |
| Escherichia coli | 3) Daily visual inspection of bioreactor samples for |
| Staphylococcus epidermidis | presence of media turbidity |
| 1. | 4) Note time of detection by Raman, Plating, Visual |

### Results

Currently, daily bioreactor samples are collected on the process floor where they need to be transferred to the QC microbiology group for spread or pour plate analysis. The time between sample collection and actual testing can be up to 6 hours, can require multiple agar plates which are prone to cross contamination and require at least an overnight incubation before any initial CFU counts can be detected. The in-line Raman method described in this study for bioreactor microbial contamination monitoring addresses these conventional plating limitations.

The parallel study results presented here met all the acceptance criteria outlined in Table 9 below demonstrating the ability of an in-line Raman method for the continuous automated, non-invasive, non-destructive direct detection of actively growing microbial contamination in mammalian cell culture bioreactors compared to the current daily conventional plating method. The Raman method successfully detected the presence of all test microorganisms spiked into the media only and cell culture bioreactors and recovered by parallel spread plating. In addition, the Raman method described in this study allows for increased data integrity and a faster time-to-detection compared to the conventional plate count CFU results while providing decision equivalence in bioreactor microbial contamination monitoring.

A visual representation of each spiking study with offline parallel plating is shown in FIG. 1.

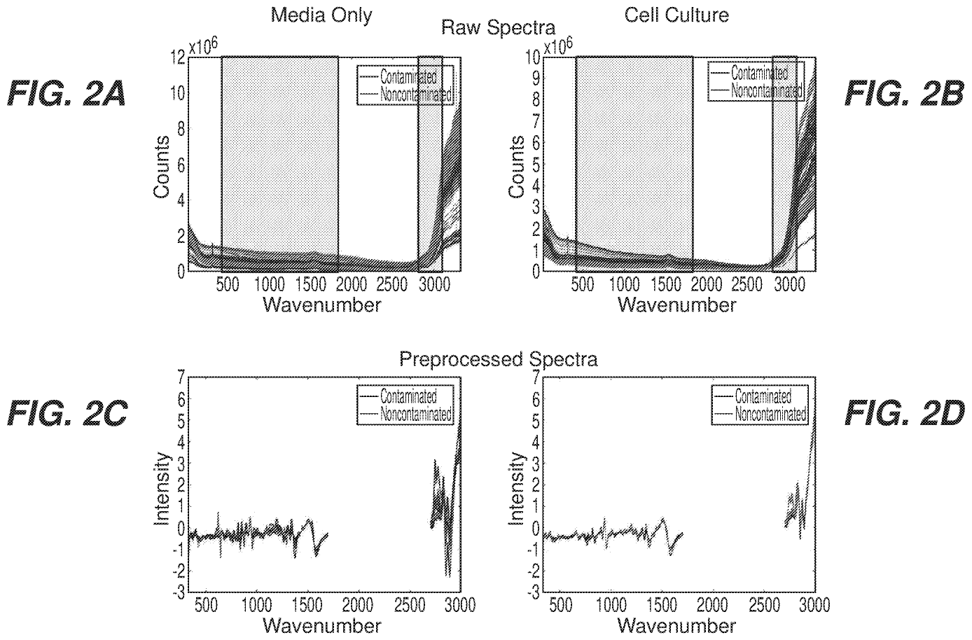

A typical spectrum (before and after pre-processing) during a bioreactor media only and cell culture processes with relevant wavenumber regions highlighted is shown in FIG. 2.

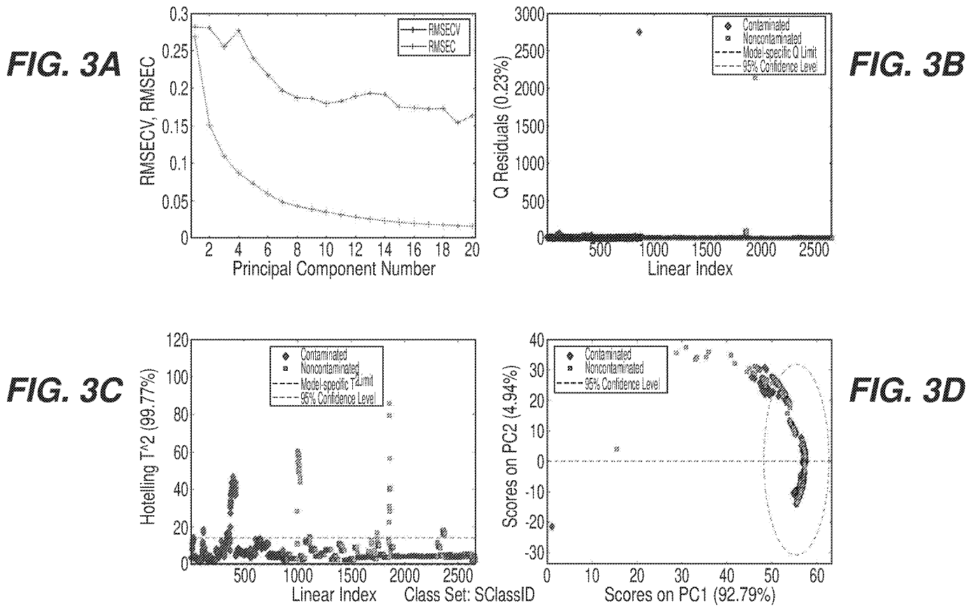

A principal component analysis (PCA) media-only model overview is shown in FIGS. 3A-3D.

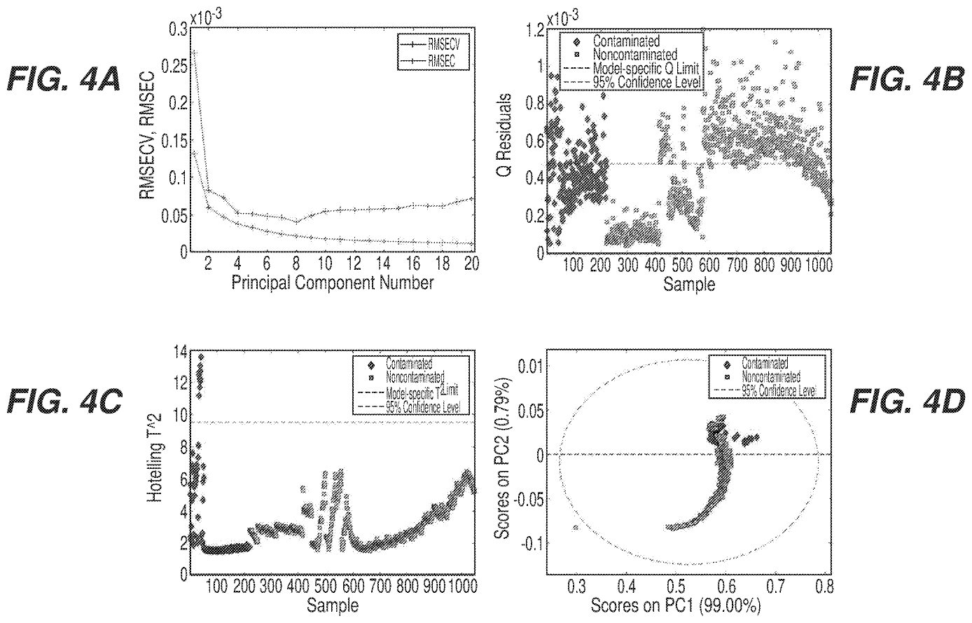

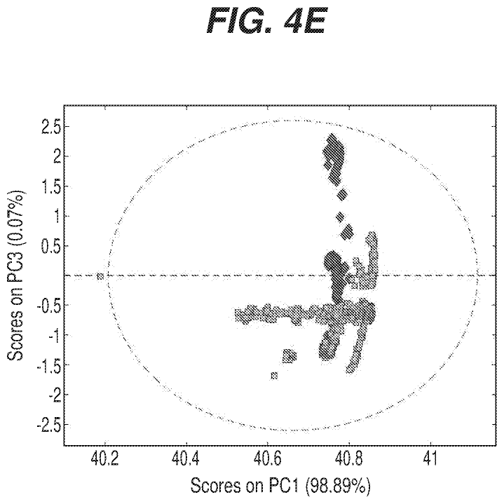

A PCA cell culture only model overview is shown in FIGS. 4A-4E.

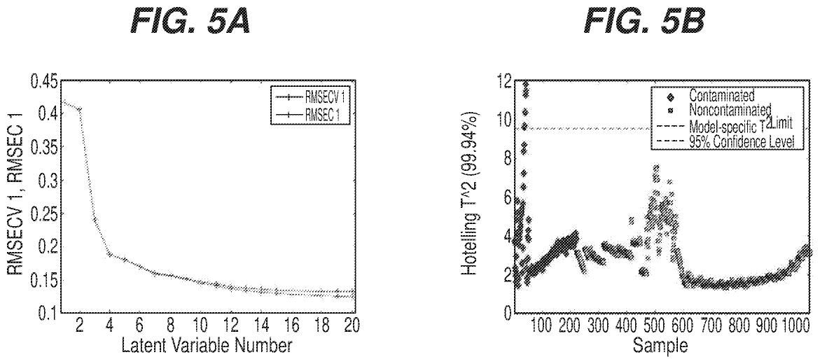

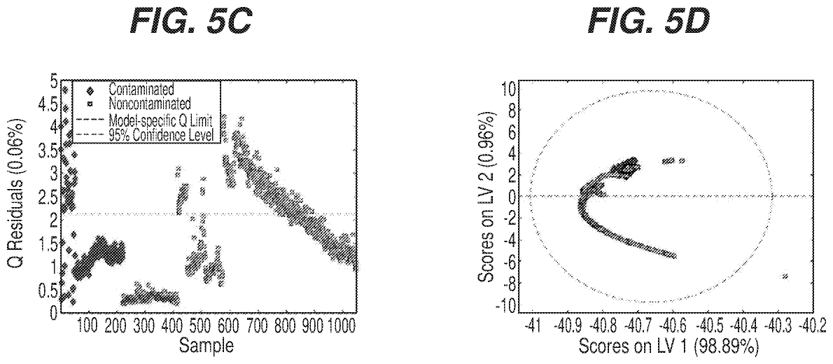

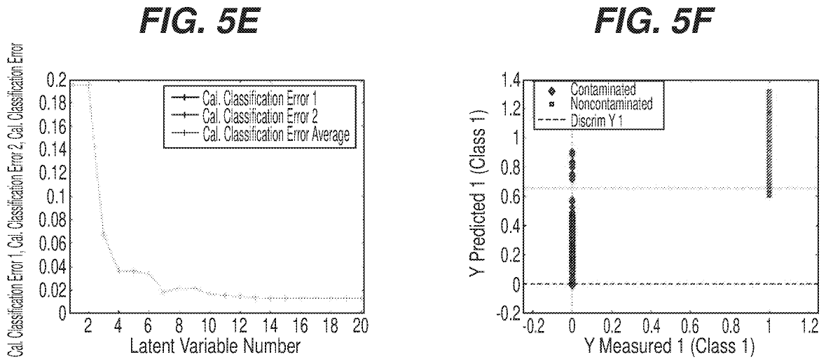

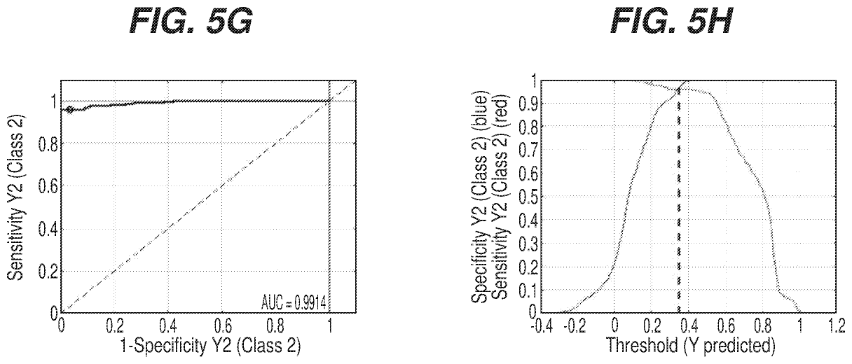

An orthogonal partial least squares discriminant analysis (OPLS-DA) cell culture model overview is shown in FIGS. 5A-5H.

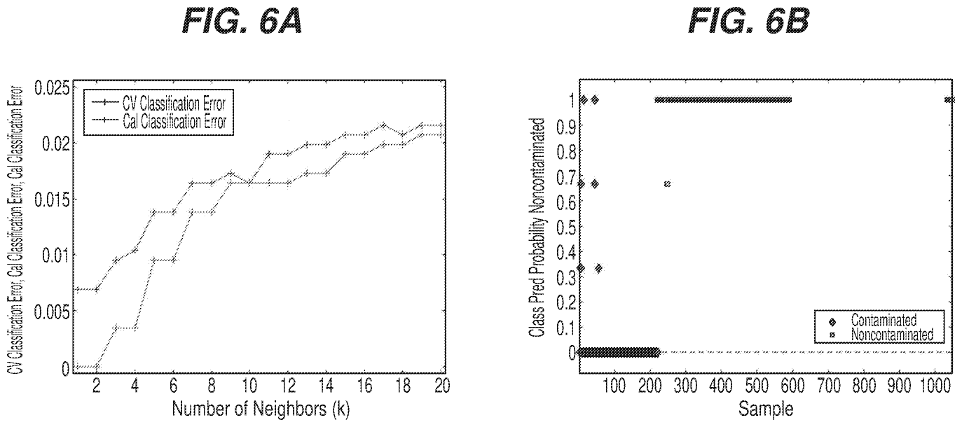

A k-nearest neighbors (KNN) cell culture model overview is shown in FIGS. 6A-6B.

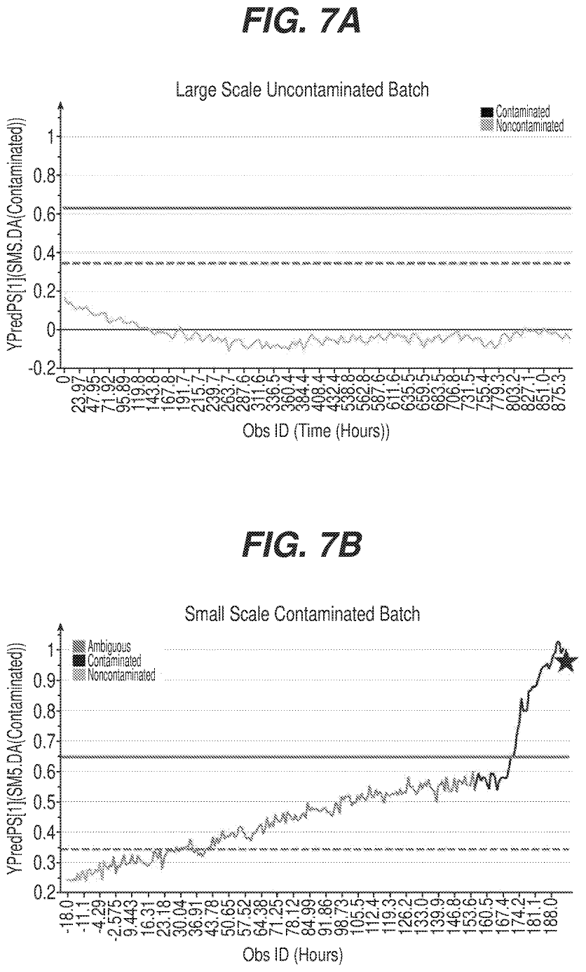

A contamination trajectory predicted with the OPLS-DA cell culture model for A) uncontaminated manufacturing scale batch and B) contaminated reduced scale batch from CTSS is shown in FIG. 7.

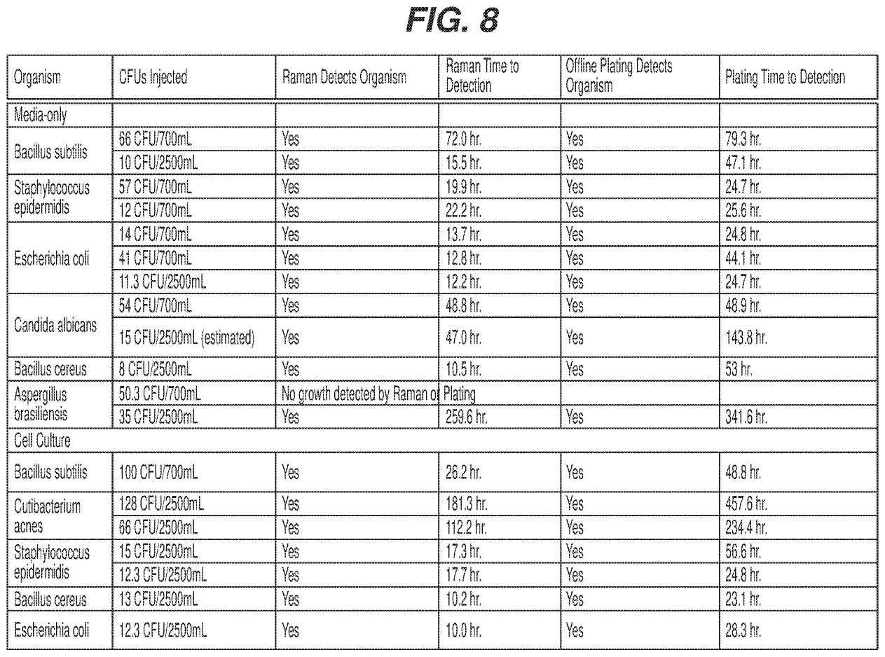

The successful Raman method bioreactor microbial contamination monitoring study results are summarized in FIG. 8.

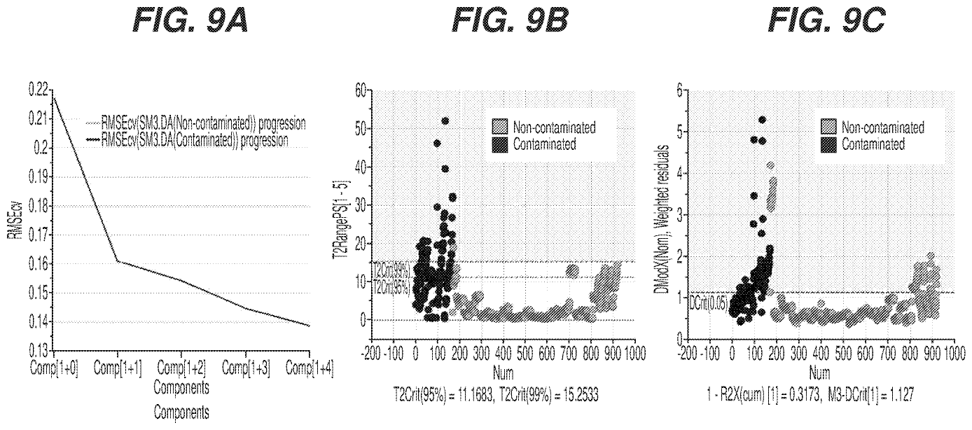

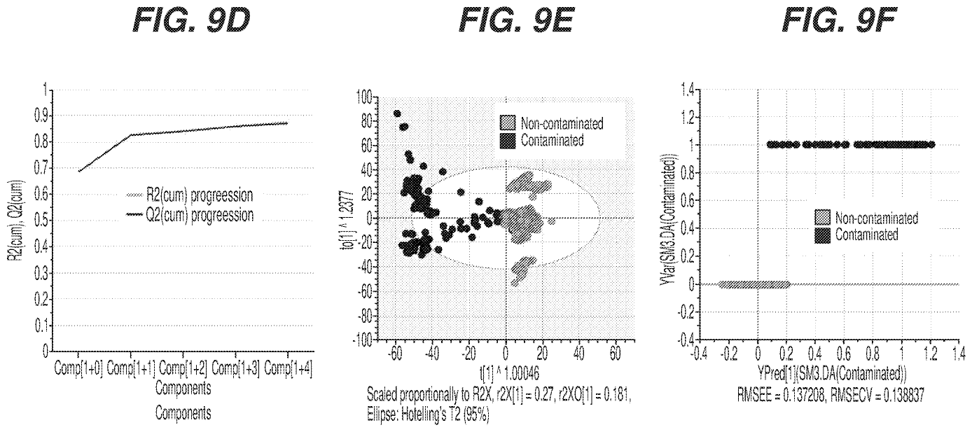

Study results using Stelara® (ustekinumab; Janssen) are summarized in FIG. 9A-FIG. 12B. FIGS. 9A-9F show a PLS-DA cell culture model overview for Stelara® (ustekinumab; Janssen) with several microbial species tested.

Exemplary methods of producing Stelara® (ustekinumab; Janssen) are illustrated in US Patent Publication No. 2020/0291107, or in US Patent Publication No. 2023/0038355, each of which is hereby incorporated by reference in its entirety.

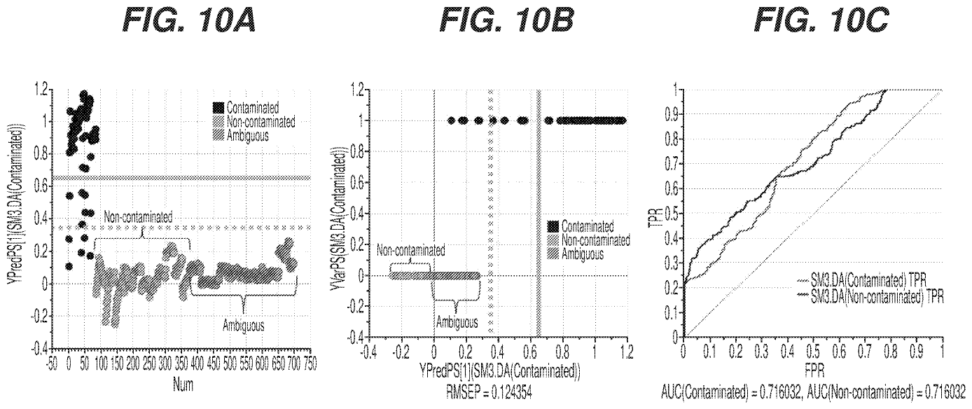

A prediction of OPLS-DA contamination model on CTSS with ambiguous transition spectra for Stelara® (ustekinumab; Janssen) with several microbial species tested is shown in FIGS. 10A-10C.

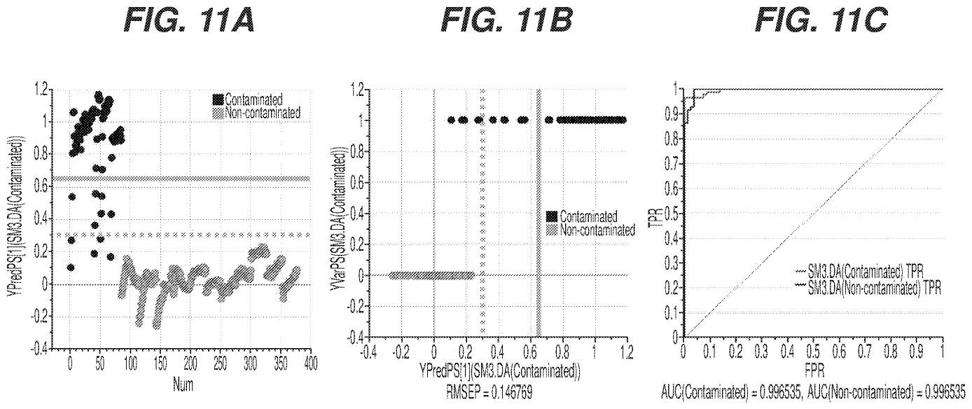

A prediction of OPLS-DA contamination model on CTSS without ambiguous transition spectra for Stelara® (ustekinumab; Janssen) with several microbial species tested is shown in FIGS. 11A-11C.

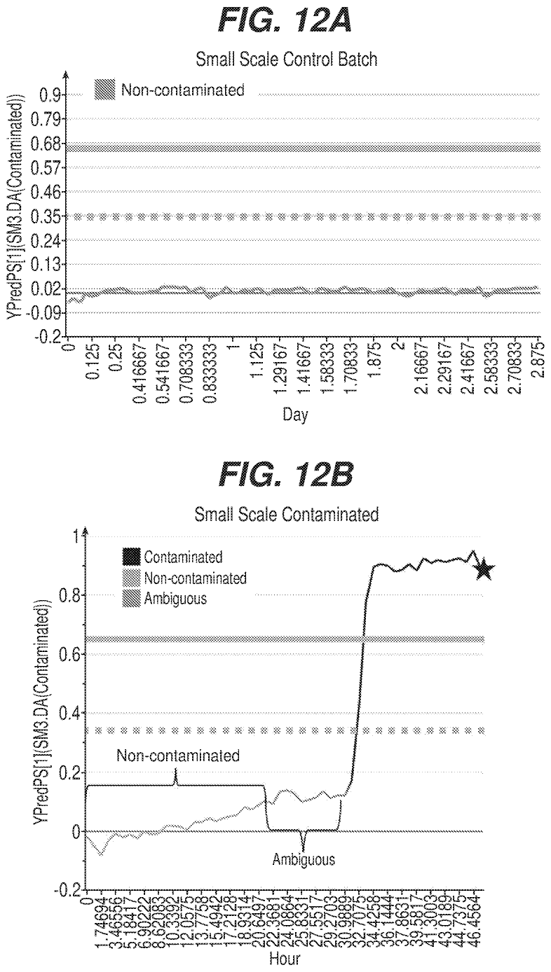

A batch trajectory prediction for Stelara® (ustekinumab; Janssen) for A) uncontaminated reduced scale batch and B) contaminated reduced scale batch from CTSS containing Bacillus cereus is shown in FIGS. 12A-12B.

A novel Raman method is presented in this study demonstrating the in-line, continuous, automated, non-invasive, non-destructive direct detection of actively growing microbial contamination in mammalian cell culture bioreactors. This Raman method was demonstrated to contain decision equivalence compared to the current daily conventional plating method but without the current limitations introduced by conventional sampling interventions.

TABLE 3
Cell Culture Bioreactor Designs

| | Bioreactor Spiking Inoculum Target Population Counts |
|---|---|
| | (colony-forming units [CFU]) |
| | ^{~} 15 CFU (^{~}100 CFU used for Bacillus subtilis and |
| Test Microorganisms | Cutibacterium acnes) |
| Bacillus cereus | 1) Bioreactors spiked with approximately 15 CFU |
| Bacillus subtilis | (^{~}100 CFU) of each designated microorganism |
| Cutibacterium acnes | 2) Daily triplicate 0.5 mL spread plating of bioreactor |
| Escherichia coli | samples performed in parallel |
| Staphylococcus epidermidis | 3) Note time of detection by Raman and Plating |

TABLE 4
Spectral Pretreatments

| | Model | | Wavelength Range |
|---|---|---|---|
| Model | Type | Preprocessing | Evaluated |
| Media | PCA | 1^{st} derivative S-G, 31 point | 425-1800, 2800-3100 |
| Only | | smoothing and SNV | cm⁻¹ |
| Cell | PCA | 1^{st} derivative S-G, 31 point | 425-1800, 2800-3100 |
| Culture | | smoothing and SNV | cm⁻¹ |
| Cell | PLS-DA | 1^{st} derivative S-G, 31 point | 425-1800, 2800-3100 |
| Culture | | smoothing and SNV | cm⁻¹ |
| Cell | KNN | 1^{st} derivative S-G, 31 point | 425-1800, 2800-3100 |
| Culture | | smoothing and SNV | cm⁻¹ |

Cell culture: mouse Sp2/0 cell line

TABLE 5
Final Models

| | | Number of | Number of | |
|---|---|---|---|---|
| | Model | Noncontaminated | Contaminated | Number of |
| Model | Type | Samples | Samples | PC/LV/NNs |
| Media Only | PCA | 1125 | 579 | 8 |
| Cell Culture | PCA | 1125 | 579 | 4 |
| Cell Culture | PLS-DA | 1125 | 579 | 4 |
| Cell Culture | KNN | 1125 | 579 | 3 |

Cell culture: mouse Sp2/0 cell line

TABLE 6
PCA Cell Culture Model Metrics

| Principal | Eigenvalue | | % Variance | | |
|---|---|---|---|---|---|
| Component | of Cov(X) | % Variance | Cumulative | RMSEC | RMSECV |
| 1 | 1.65e+03 | 98.89 | 98.89 | 0.1055 | 0.1698 |
| 2 | 1.61e+01 | 0.97 | 99.85 | 0.03834 | 0.0419 |
| 3 | 1.13e+00 | 0.7 | 99.92 | 0.02823 | 0.03193 |
| 4 | 3.54e−01 | 0.2 | 99.94 | 0.02419 | 0.02768 |

TABLE 7
PLS-DA Confusion Matrix. Sensitivity = 0.96, Specificity = 0.96

| Class: | Noncontaminated | Contaminated |
|---|---|---|
| Noncontaminated | 801 | 9 |
| Predicted | | |
| Contaminated | 28 | 211 |
| Predicted | | |
| Unassigned | 0 | 0 |
| Predicted | | |

TABLE 8
KNN Confusion Matrix. Sensitivity = 1.00, Specificity = 0.99

| Class: | Noncontaminated | Contaminated |
|---|---|---|
| Noncontaminated | 829 | 4 |
| Predicted | | |
| Contaminated | 0 | 216 |
| Predicted | | |
| Unassigned | 0 | 0 |
| Predicted | | |

TABLE 9
Summary of method performance acceptance criteria

| Parameter | Performance |
|---|---|
| Specificity | Successfully distinguished between a contamination event and |
| | normal cell culture |
| LOD | The minimum number of organisms that can be detected but not |
| | necessarily quantified. The Raman method successfully detected |
| | low starting microorganism contamination levels of < 1CFU/mL |
| | in the test bioreactors |
| Robustness | Robustness: The ability of the Raman method to detect |
| | contamination throughout duration of the cell culture batch in |
| | which the matrix is constantly changing with respect to total cell |
| | density, metabolites, titer, and feed. Will be addressed in future |
| | parallel in-process testing |
| Ruggedness | The degree of precision of test results from replicate bioreactors |
| | operating under the same conditions. Will be addressed in |
| | future parallel in-process testing |
| Equivalency | Decision equivalence is established by comparing the time to |
| | detect contamination. The Raman method was equivalent or |
| | superior to the current compendial method |
| Scale | Applicable to 1L, 3L, 5L, 10L, 1000L, 2000L or larger scale |
| | bioreactors |
| Application | Relevant, for example, for Kaiser RXN2/4 Raman Analyzers |

### Embodiments

The following exemplary embodiments further describe optional aspects of the presently disclosed technology and are part of the Detailed Description. These exemplary embodiments are set forth in a format substantially akin to claims (each with numerical designations followed by a capital letter), although they are not technically claims of the present application. The following exemplary embodiments refer to each other in dependent relationships as “embodiments” instead of “claims.”
1A. A method for detecting a microbe in a sample comprising: measuring a Raman spectrum of the sample.
2A. The method of embodiment 1A, wherein the sample is exposed to a coherent light source prior to measuring the Raman spectrum of the sample.
3A. The method of embodiment, 1A, wherein the Raman spectrum is measured using a Raman spectrometer.
4A. The method of embodiment 3A, wherein the Raman spectrometer comprises a laser.
5A. The method of embodiment 4A, wherein the laser is a multimode diode laser.
6A. The method of embodiment 4A or embodiment 5A, wherein the laser wavelength is from 300 nm to 1200 nm.
7A. The method of embodiment 6A, wherein the laser wavelength is 785 nm.
8A. The method of any one of embodiments, 1A-7A, wherein the sample is in a bioreactor, a harvest tank, a chromatography skid, a chromatography column, an ultrafiltration (UF) skid, a diafiltration (DF) skid, a UF/DF skid, a fill finish tank or an incubator.
9A. The method of any one of embodiments 1A-8A, wherein the Raman spectrum of the sample is compared to the Raman spectrum of a control sample.
10A. The method of any one of embodiments 1A-9A, wherein the Raman spectrum of the sample is monitored over a period of time.
11A. The method of any one of embodiments 1A-10A, wherein the monitoring is continuous.
12A. The method of any one of embodiments 1A-11A, wherein the monitoring is non-invasive and/or non-destructive.
13A. The method of any one of embodiments 1A-12A, wherein the sample has been contacted with an antibody to the microbe.
14A. The method of any one of embodiments 1A-13A, wherein the sample has been contacted with a fluorescent agent or a bioluminescent agent.
15A. The method of any one of embodiments 1A-14A, further comprising detecting fluorescence or bioluminescence of the sample.
16A. The method of any one of embodiments 1A-15A, wherein the microbe is Cutibacterium acnes, Staphylococcus aureus, Aspergillus brasiliensis, Candida albicans, Bacillus subtilis, Escherichia coli, Pseudomonas aeruginosa, Staphylococcus epidermidis, Streptococcus pyogenes, Micrococcus luteus, Burkholderia cepacia, Salmonella typhimurium, or Bacillus cereus.
17A. The method of any one of embodiments 1A-16A, wherein the measuring of the Raman spectrum is automated.
18A. The method of any one of embodiments 1A-17A, wherein the Raman spectrum is subjected to data analysis.
19A. The method of any one of embodiments 1A-18A, wherein data analysis comprises generating one or more models based on the obtained Raman spectrum data that correlate levels of microbe with the obtained Raman spectrum data.
20A. The method of any one of embodiments 1A-19A, wherein the one or more models are regression models.
21A. The method of any one of embodiments 1A-20A, wherein data analysis of the Raman spectrum comprises modeling performed using PCA-X, OPLS, KNN, OPLS-DA, PLS-DA, or combinations thereof.
22A. The method of any one of embodiments 1A-21A, wherein the microbe is actively growing.
1B. A method for continuous monitoring for microbial presence in a sample comprising: measuring a Raman spectrum of the sample.
2B. The method of embodiment 1B, wherein the Raman spectrum of the sample is monitored over a period of time.
3B. The method of embodiment 1B or embodiment 2B, wherein the monitoring is non-invasive and/or non-destructive.
4B. The method of any one of embodiments 1B-3B, wherein the sample is exposed to a coherent light source prior to measuring the Raman spectrum of the sample.
5B. The method of any one of embodiments 1B-4B, wherein the Raman spectrum is measured using a Raman spectrometer.
6B. The method of any one of embodiments 1B-5B, wherein the Raman spectrometer comprises a laser.
7B. The method of embodiment 6B, wherein the laser is a multimode diode laser.
8B. The method of embodiment 6B or embodiment 7B, wherein the laser wavelength is from 300 nm to 1200 nm.
9B. The method of embodiment 8B, wherein the laser wavelength is 785 nm.
10B. The method of any one of embodiments 1B-9B, wherein the sample is in a bioreactor, a harvest tank, a chromatography skid, a chromatography column, an ultrafiltration (UF) skid, a diafiltration (DF) skid, a UF/DF skid, a fill finish tank or an incubator.
11B. The method of any one of embodiments 1B-10B, wherein the Raman spectrum of the sample is compared to the Raman spectrum of a control sample.
12B. The method of any one of embodiments 1B-11B, wherein the sample has been contacted with an antibody to the microbe.
13B. The method of any one of embodiments 1B-12B, wherein the sample has been contacted with a fluorescent agent or a bioluminescent agent.
14B. The method of any one of embodiments 1B-13B, further comprising detecting fluorescence or bioluminescence of the sample.
15B. The method of any one of embodiments 1B-14B, wherein the microbe is Cutibacterium acnes, Staphylococcus aureus, Aspergillus brasiliensis, Candida albicans, Bacillus subtilis, Escherichia coli, Pseudomonas aeruginosa, Staphylococcus epidermidis, Streptococcus pyogenes, Micrococcus luteus, Burkholderia cepacia, Salmonella typhimurium, or Bacillus cereus.
16B. The method of any one of embodiments 1B-15B, wherein the measuring of the Raman spectrum is automated.
17B. The method of any one of embodiments 1B-16B, wherein the Raman spectrum is subjected to data analysis.
18B. The method of any one of embodiments 1B-17B, wherein data analysis comprises generating one or more models based on the obtained Raman spectrum data that correlate levels of microbe with the obtained Raman spectrum data.
19B. The method of any one of embodiments 1B-18B, wherein the one or more models are regression models
20B. The method of any one of embodiments 1B-19B, wherein data analysis of the Raman spectrum comprises discriminant modeling performed using PCA-X, OPLS, KNN, OPLS-DA, PLS-DA, or combinations thereof.
21B. The method of any one of embodiments 1B-20B, wherein the microbe is actively growing.
1C. A method for assaying a test agent comprising: adding a test agent to a sample comprising a microbe; and measuring a Raman spectrum of the sample.
2C. The method of embodiment 1C, wherein the Raman spectrum is measured using a Raman spectrometer.
3C. The method of embodiment 2C, wherein the Raman spectrometer comprises a laser.
4C. The method of embodiment 3C, wherein the laser is a multimode diode laser.
5C. The method of embodiment 3C or embodiment 4C, wherein the laser wavelength is from 300 nm to 1200 nm.
6C. The method of embodiment 5C, wherein the laser wavelength is 785 nm.
7C. The method of any one of embodiments 1C-6C, wherein the sample is in a bioreactor, a harvest tank, a chromatography skid, a chromatography column, an ultrafiltration (UF) skid, a diafiltration (DF) skid, a UF/DF skid, a fill finish tank or an incubator.
8C. The method of any one of embodiments 1C-7C, wherein the Raman spectrum of the sample is compared to the Raman spectrum of a control sample.
9C. The method of any one of embodiments 1C-8C, wherein the Raman spectrum of the sample is monitored over a period of time.
10C. The method of any one of embodiments 1C-9C, wherein the monitoring is continuous.
11C. The method of any one of embodiments 1C-10C, wherein the monitoring is non-invasive and/or non-destructive.
12C. The method of any one of embodiments 1C-11C, wherein the test agent is a compound, a supplement, an antibiotic, a bacteriophage, or a combination thereof.
13C. The method of any one of embodiments 1C-12C, wherein the sample has been contacted with an antibody to the microbe.
14C. The method of any one of embodiments 1C-13C, wherein the sample has been contacted with a fluorescent agent or a bioluminescent agent.
15C. The method of any one of embodiments 1C-14C, further comprising detecting fluorescence or bioluminescence of the sample.
16C. The method of any one of embodiments 1C-15C, wherein the microbe is Cutibacterium acnes, Staphylococcus aureus, Aspergillus brasiliensis, Candida albicans, Bacillus subtilis, Escherichia coli, Pseudomonas aeruginosa, Staphylococcus epidermidis, or Bacillus cereus.
17C. The method of any one of embodiments 1C-16C, wherein the measuring of the Raman spectrum is automated.
18C. The method of any one of embodiments 1C-17C, wherein the Raman spectrum is subjected to data analysis.
19C. The method of any one of embodiments 1C-18C, wherein data analysis comprises generating one or more models based on the obtained Raman spectrum data that correlate levels of microbe with the obtained Raman spectrum data.
20C. The method of any one of embodiments 1C-19C, wherein the one or more models are regression models.
21C. The method of any one of embodiments 1C-20C, wherein data analysis of the Raman spectrum comprises discriminant modeling performed using PCA-X, OPLS, KNN, OPLS-DA, PLS-DA, or combinations thereof.
22C. The method of any one of embodiments 1C-21C, wherein the microbe is actively growing.
1D. A system for detecting a microbe in a sample comprising: a means for measuring a Raman spectrum of the sample.
2D. The system of embodiment 1D, further comprising a coherent light source.
3D. The system of embodiment 1D, wherein the means for measuring a Raman spectrum is a Raman spectrometer.
4D. The system of embodiment 3D, wherein the Raman spectrometer comprises a laser.
5D. The system of embodiment 4D, wherein the laser is a multimode diode laser.
6D. The system of embodiment 4D or 5D, wherein the laser wavelength is from 300 nm to 1200 nm.
7D. The system of embodiment 6D, wherein the laser wavelength is 785 nm.
8D. The system of any one of embodiments 1D-7D, wherein the sample is in a bioreactor, a harvest tank, a chromatography skid, a chromatography column, an ultrafiltration (UF) skid, a diafiltration (DF) skid, a UF/DF skid, a fill finish tank or an incubator.
9D. The system of any one of embodiments 1D-8D, wherein the Raman spectrum of the sample is compared to the Raman spectrum of a control sample.
10D. The system of any one of embodiments 1D-9D, wherein the Raman spectrum of the sample is monitored over a period of time.
11D. The system of any one of embodiments 1D-10D, wherein the monitoring is continuous.
12D. The system of any one of embodiments 1D-11D, wherein the monitoring is non-invasive and/or non-destructive.
13D. The system of any one of embodiments 1D-12D, wherein the sample has been contacted with an antibody to the microbe.
14D. The system of any one of embodiments 1D-13D, wherein the sample has been contacted with a fluorescent agent or a bioluminescent agent.
15D. The system of any one of embodiments 1D-14D, further comprising detecting fluorescence or bioluminescence of the sample.
16D. The system of any one of embodiments 1D-15D, wherein the microbe is Cutibacterium acnes, Staphylococcus aureus, Aspergillus brasiliensis, Candida albicans, Bacillus subtilis, Escherichia coli, Pseudomonas aeruginosa, Staphylococcus epidermidis, Streptococcus pyogenes, Micrococcus luteus, Burkholderia cepacia, Salmonella typhimurium, or Bacillus cereus.
17D. The system of any one of embodiments 1D-16D, wherein the measuring of the Raman spectrum is automated.
18D. The system of any one of embodiments 1D-17D, wherein the Raman spectrum is subjected to data analysis.
19D. The system of any one of embodiments 1D-18D, wherein data analysis comprises generating one or more models based on the obtained Raman spectrum data that correlate levels of microbe with the obtained Raman spectrum data.
20D. The system of any one of embodiments 1D-19D, wherein the one or more models are regression models.
21D. The system of any one of embodiments 1D-20D, wherein data analysis of the Raman spectrum comprises modeling performed using PCA-X, OPLS, KNN, OPLS-DA, PLS-DA, or combinations thereof.
22D. The system of any one of embodiments 1D-21D, wherein the microbe is actively growing.
1E. A system for continuous monitoring for microbial presence in a sample comprising: a means for measuring a Raman spectrum of the sample.
2E. The system of embodiment 1E, wherein the Raman spectrum of the sample is monitored over a period of time.
3E. The system of embodiment 1E or embodiment 2E, wherein the monitoring is non-invasive and/or non-destructive.
4E. The system of any one of embodiments 1E-3E, wherein the sample is exposed to a coherent light source prior to measuring the Raman spectrum of the sample.
5E. The system of any one of embodiments 1E-4E, wherein the Raman spectrum is measured using a Raman spectrometer.
6E. The system of any one of embodiments 1E-5E, wherein the Raman spectrometer comprises a laser.
7E. The system of embodiment 6E, wherein the laser is a multimode diode laser.
8E. The system of embodiment 6E or embodiment 7E, wherein the laser wavelength is from 300 nm to 1200 nm.
9E. The system of embodiment 8E, wherein the laser wavelength is 785 nm.
10E. The system of any one of embodiments 1E-9E, wherein the sample is in a bioreactor, a harvest tank, a chromatography skid, a chromatography column, an ultrafiltration (UF) skid, a diafiltration (DF) skid, a UF/DF skid, a fill finish tank or an incubator.
11E. The system of any one of embodiments 1E-10E, wherein the Raman spectrum of the sample is compared to the Raman spectrum of a control sample.
12E. The system of any one of embodiments 1E-11E, wherein the sample has been contacted with an antibody to the microbe.
13E. The system of any one of embodiments 1E-12E, wherein the sample has been contacted with a fluorescent agent or a bioluminescent agent.
14E. The system of any one of embodiments 1E-13E, further comprising detecting fluorescence or bioluminescence of the sample.
15E. The system of any one of embodiments 1E-14E, wherein the microbe is Cutibacterium acnes, Staphylococcus aureus, Aspergillus brasiliensis, Candida albicans, Bacillus subtilis, Escherichia coli, Pseudomonas aeruginosa, Staphylococcus epidermidis, Streptococcus pyogenes, Micrococcus luteus, Burkholderia cepacia, Salmonella typhimurium, or Bacillus cereus.
16E. The system of any one of embodiments 1E-15E, wherein the measuring of the Raman spectrum is automated.
17E. The system of any one of embodiments 1E-16E, wherein the Raman spectrum is subjected to data analysis.
18E. The system of any one of embodiments 1E-17E, wherein data analysis comprises generating one or more models based on the obtained Raman spectrum data that correlate levels of microbe with the obtained Raman spectrum data.
19E. The system of any one of embodiments 1E-18E, wherein the one or more models are regression models.
20E. The system of any one of embodiments 1E-19E, wherein data analysis of the Raman spectrum comprises discriminant modeling performed using PCA-X, OPLS, KNN, OPLS-DA, PLS-DA, or combinations thereof.
21E. The system of any one of embodiments 1E-20E, wherein the microbe is actively growing.
1F. A system for assaying a test agent comprising: a means for measuring a Raman spectrum of a sample to which a test agent has been added.
2F. The system of embodiment 1F, further comprising a coherent light source.
3F. The system of embodiment 1F, wherein the means for measuring a Raman spectrum is a Raman spectrometer.
4F. The system of embodiment 3F, wherein the Raman spectrometer comprises a laser.
5F. The system of embodiment 4F, wherein the laser is a multimode diode laser.
6F. The system of embodiment 4F or embodiment 5F, wherein the laser wavelength is from 300 nm to 1200 nm.
7F. The system of embodiment 6F, wherein the laser wavelength is 785 nm.
8F. The system of any one of embodiments 1F-7F, wherein the sample is in a bioreactor, a harvest tank, a chromatography skid, a chromatography column, an ultrafiltration (UF) skid, a diafiltration (DF) skid, a UF/DF skid, a fill finish tank or an incubator.
9F. The system of any one of embodiments 1F-8F, wherein the Raman spectrum of the sample is compared to the Raman spectrum of a control sample.
10F. The system of any one of embodiments 1F-9F, wherein the Raman spectrum of the sample is monitored over a period of time.
11F. The system of any one of embodiments 1F-10F, wherein the monitoring is continuous.
12F. The system of any one of embodiments 1F-11F, wherein the monitoring is non-invasive and/or non-destructive.
13F. The system of any one of embodiments 1F-12F, wherein the sample has been contacted with an antibody to the microbe.
14F. The system of any one of embodiments 1F-13F, wherein the sample has been contacted with a fluorescent agent or a bioluminescent agent.
15F. The system of any one of embodiments 1F-14F, further comprising detecting fluorescence or bioluminescence of the sample.
16F. The system of any one of embodiments 1F-15F, wherein the microbe is Cutibacterium acnes, Staphylococcus aureus, Aspergillus brasiliensis, Candida albicans, Bacillus subtilis, Escherichia coli, Pseudomonas aeruginosa, Staphylococcus epidermidis, or Bacillus cereus.
17F. The system of any one of embodiments 1F-16F, wherein the measuring of the Raman spectrum is automated.
18F. The system of any one of claims 1F-17F, wherein the Raman spectrum is subjected to data analysis.
19F. The system of any one of embodiments 1F-18F, wherein data analysis comprises generating one or more models based on the obtained Raman spectrum data that correlate levels of microbe with the obtained Raman spectrum data.
20F. The system of any one of embodiments 1F-19F, wherein the one or more models are regression models.
21F. The system of any one of embodiments 1F-20F, wherein data analysis of the Raman spectrum comprises modeling performed using PCA-X, OPLS, KNN, OPLS-DA, PLS-DA, or combinations thereof.
22F. The system of any one of embodiments 1F-21F, wherein the microbe is actively growing.

### REFERENCES

1. USP-NF <61> Microbiological Examination of Nonsterile Products Microbial Enumeration Tests.
2. 2.6.12. MICROBIOLOGICAL EXAMINATION OF NON-STERILE PRODUCTS (TOTAL VIABLE AEROBIC COUNT). Pharmacopeia, E., Ed.
3. Rapid Detection of Bacteria and Viruses in Bioprocess samples: Justification, regulation, requirements and technologies—How can industry achieve broad adoption?; Biophorum Operations Group: 2019.
4. Vargas, J. M.; Nielsen, S.; Cárdenas, V.; Gonzalez, A.; Aymat, E. Y.; Almodovar, E.; Classe, G.; Colón, Y.; Sanchez, E.; Romañach, R. J., Process analytical technology in continuous manufacturing of a commercial pharmaceutical product. International Journal of Pharmaceutics 2018, 538 (1), 167-178.
5. Krogmeier, S.; Pritchard, J.; Dokou, E.; Miles, S.; Connelly, G.; Medendorp, J.; Bourland, M.; Swinney, K., Chapter 16-Orkambi: a continuous manufacturing approach to process development at Vertex. In How to Design and Implement Powder-To-Tablet Continuous Manufacturing Systems, Muzzio, F. J.; Oka, S., Eds. Academic Press: 2022; pp 383-396.
6. Abu-Absi, N. R.; Kenty, B. M.; Cuellar, M. E.; Borys, M. C.; Sakhamuri, S.; Strachan, D. J.; Hausladen, M. C.; Li, Z. J., Real time monitoring of multiple parameters in mammalian cell culture bioreactors using an in-line Raman spectroscopy probe. Biotechnol Bioeng 2011, 108 (5), 1215-21.
7. Berry, B.; Moretto, J.; Matthews, T.; Smelko, J.; Wiltberger, K., Cross-scale predictive modeling of CHO cell culture growth and metabolites using Raman spectroscopy and multivariate analysis. Biotechnol Prog 2015, 31 (2), 566-77.
8. Mehdizadeh, H.; Lauri, D.; Karry, K. M.; Moshgbar, M.; Procopio-Melino, R.; Drapeau, D., Generic Raman-based calibration models enabling real-time monitoring of cell culture bioreactors. Biotechnol Prog 2015, 31 (4), 1004-13.
9. Sellick, C. A.; Hansen, R.; Jarvis, R. M.; Maqsood, A. R.; Stephens, G. M.; Dickson, A. J.; Goodacre, R., Rapid monitoring of recombinant antibody production by mammalian cell cultures using fourier transform infrared spectroscopy and chemometrics. Biotechnol Bioeng 2010, 106 (3), 432-42.
10. Helgers, H.; Schmidt, A.; Lohmann, L. J.; Vetter, F. L.; Juckers, A.; Jensch, C.; Mouellef, M.; Zobel-Roos, S.; Strube, J., Towards Autonomous Operation by Advanced Process Control—Process Analytical Technology for Continuous Biologics Antibody Manufacturing. Processes 2021, 9 (1), 172.
11. Moore, B.; Sanford, R.; Zhang, A., Case study: The characterization and implementation of dielectric spectroscopy (biocapacitance) for process control in a commercial GMP CHO manufacturing process. Biotechnology Progress 2019, 35 (3), e2782.
12. Reyes, S. J.; Durocher, Y.; Pham, P. L.; Henry, O., Modern Sensor Tools and Techniques for Monitoring, Controlling, and Improving Cell Culture Processes. Processes 2022, 10 (2), 189.
13. Zhang, A.; Tsang, V. L.; Moore, B.; Shen, V.; Huang, Y.-M.; Kshirsagar, R.; Ryll, T., Advanced process monitoring and feedback control to enhance cell culture process production and robustness. Biotechnology and Bioengineering 2015, 112 (12), 2495-2504.
14. André, S.; Cristau, L. S.; Gaillard, S.; Devos, O.; Calvosa, É.; Duponchel, L., In-line and real-time prediction of recombinant antibody titer by in situ Raman spectroscopy. Analytica Chimica Acta 2015, 892, 148-152.
15. Wei, B.; Woon, N.; Dai, L.; Fish, R.; Tai, M.; Handagama, W.; Yin, A.; Sun, J.; Maier, A.; McDaniel, D.; Kadaub, E.; Yang, J.; Saggu, M.; Woys, A.; Pester, O.; Lambert, D.; Pell, A.; Hao, Z.; Magill, G.; Yim, J.; Chan, J.; Yang, L.; Macchi, F.; Bell, C.; Deperalta, G.; Chen, Y., Multi-attribute Raman spectroscopy (MARS) for monitoring product quality attributes in formulated monoclonal antibody therapeutics. MAbs 2022, 14 (1), 2007564.
16. Hu, Q.; Jiang, B.; Liu, D.; Tang, X.; Daly, T.; Shameem, M., Chapter 14: Practical Considerations in High Concentration Formulation Development for Monoclonal Antibody Drug Products. In Development of Biopharmaceutical Drug-Device Products, Jameel, F.; Skoug, J. W.; Nesbitt, R. R., Eds. Springer International Publishing: Cham, 2020; pp 343-372.
17. Thakur, G.; Hebbi, V.; Rathore, A. S., Near Infrared Spectroscopy as a PAT tool for monitoring and control of protein and excipient concentration in ultrafiltration of highly concentrated antibody formulations. International Journal of Pharmaceutics 2021, 600, 120456.
18. Zhang, C.; Springall, J. S.; Wang, X.; Barman, I., Rapid, quantitative determination of aggregation and particle formation for antibody drug conjugate therapeutics with label-free Raman spectroscopy. Analytica Chimica Acta 2019, 1081, 138-145.
19. Harz, M.; Stöckel, S.; Ciobota, V.; Cialla, D.; Rösch, P.; Popp, J., Applications of Raman Spectroscopy to Virology and Microbial Analysis. In Emerging Raman Applications and Techniques in Biomedical and Pharmaceutical Fields, Matousek, P.; Morris, M. D., Eds. Springer Berlin Heidelberg: Berlin, Heidelberg, 2010; pp 439-463.
20. Ashton, L.; Lau, K.; Winder, C. L.; Goodacre, R., Raman spectroscopy: lighting up the future of microbial identification. Future Microbiology 2011, 6 (9), 991-997.
21. Maruthamuthu, M. K.; Raffiee, A. H.; De Oliveira, D. M.; Ardekani, A. M.; Verma, M. S., Raman spectra-based deep learning: A tool to identify microbial contamination. MicrobiologyOpen 2020, 9 (11), e1122.
22. Bugno, A.; Saes, D. P. S.; Almodovar, A. A. B.; Dua, K.; Awasthi, R.; Ghisleni, D. D. M.; Hirota, M. T.; de Oliveira, W. A.; de Jesus Andreoli Pinto, T., Performance Survey and Comparison Between Rapid Sterility Testing Method and Pharmacopoeia Sterility Test. Journal of Pharmaceutical Innovation 2018, 13 (1), 27-35.
23. <USP 1223 Validation of Aternative Microbiological Methods.pdf>. Pharmocopeia, U. S., Ed.
24. EU5.1.6 AlternativeMethodsforControlofMicrobiologicalQuality. Pharmacopeia, E. U., Ed.
25. Grosso, R. A. et al. Detection of low numbers of bacterial cells in a pharmaceutical drug product using Raman spectroscopy and PLS-DA multivariate analysis. Analyst, 2022, 147:3593.

All publications and patents referred to herein are incorporated by reference. Various modifications and variations of the described subject matter will be apparent to those skilled in the art without departing from the scope and spirit of the invention. Although the invention has been described in connection with specific embodiments, it should be understood that the invention as claimed should not be unduly limited to these embodiments. Indeed, various modifications for carrying out the invention are obvious to those skilled in the art and are intended to be within the scope of the following claims.

1. A method for detecting a microbe in a sample comprising:
measuring a Raman spectrum of the sample.

2. The method of claim 1, wherein the sample is exposed to a coherent light source prior to measuring the Raman spectrum of the sample.

3. The method of claim 1, wherein the Raman spectrum is measured using a Raman spectrometer.

4. The method of claim 3, wherein the Raman spectrometer comprises a laser.

5. The method of claim 4, wherein the laser is a multimode diode laser.

6. The method of claim 4, wherein the laser wavelength is from 300 nm to 1200 nm.

7. The method of claim 6, wherein the laser wavelength is 785 nm.

8. The method of claim 1, wherein the sample is in a bioreactor, a harvest tank, a chromatography skid, a chromatography column, an ultrafiltration (UF) skid, a diafiltration (DF) skid, a UF/DF skid, a fill finish tank or an incubator.

9. The method of claim 1, wherein the Raman spectrum of the sample is compared to the Raman spectrum of a control sample.

10. The method of claim 1, wherein the Raman spectrum of the sample is monitored over a period of time.

11. The method of claim 1, wherein the monitoring is continuous.

12. The method of claim 1, wherein the monitoring is non-invasive and/or non-destructive.

13. The method of claim 1, wherein the sample has been contacted with an antibody to the microbe.

14. The method of claim 1, wherein the sample has been contacted with a fluorescent agent or a bioluminescent agent.

15. The method claim 1, further comprising detecting fluorescence or bioluminescence of the sample.

16. The method of claim 1, wherein the microbe is Cutibacterium acnes, Staphylococcus aureus, Aspergillus brasiliensis, Candida albicans, Bacillus subtilis, Escherichia coli, Pseudomonas aeruginosa, Staphylococcus epidermidis, Streptococcus pyogenes, Micrococcus luteus, Burkholderia cepacia, Salmonella typhimurium, or Bacillus cereus.

17. The method of claim 1, wherein the measuring of the Raman spectrum is automated.

18. The method of claim 1, wherein the Raman spectrum is subjected to data analysis.

19. The method of claim 1, wherein data analysis of the Raman spectrum comprises modeling performed using PCA-X, OPLS, KNN, OPLS-DA, PLS-DA, or combinations thereof.

20. The method of claim 1, wherein the microbe is actively growing.

21. A method for continuous monitoring for microbial presence in a sample comprising: measuring a Raman spectrum of the sample.

22. The method of claim 21, wherein the Raman spectrum of the sample is monitored over a period of time.

23. The method of claim 21, wherein the monitoring is non-invasive and/or nondestructive.

24. A method for assaying a test agent comprising:
adding a test agent to a sample comprising a microbe; and
measuring a Raman spectrum of the sample.

25. The method of claim 24, wherein the Raman spectrum is measured using a Raman spectrometer.

26. The method of claim 25, wherein the Raman spectrometer comprises a laser.

27. A system for detecting a microbe in a sample comprising: a means for measuring a Raman spectrum of the sample.

28. The system of claim 27, further comprising a coherent light source.

29. The system of claim 27, wherein the means for measuring a Raman spectrum is a Raman spectrometer.

30. A system for continuous monitoring for microbial presence in a sample comprising: a means for measuring a Raman spectrum of the sample.

31. The system of claim 30, wherein the Raman spectrum of the sample is monitored over a period of time.

32. The system of claim 30, wherein the monitoring is non-invasive and/or nondestructive.

33. A system for assaying a test agent comprising: a means for measuring a Raman spectrum of a sample to which a test agent has been added.

34. The system of claim 33, further comprising a coherent light source.

35. The system of claim 33, wherein the means for measuring a Raman spectrum is a Raman spectrometer.
